(19) United States
(12) Patent Application Publication
(10) Pub. No.: US 2022/0243160 A1
(43) Pub. Date: Aug. 4, 2022

(54) Method and Apparatus for Interrogating Biological Systems

(71) Applicant: Arizona Board of Regents on Behalf of the University of Arizona, Tucson, AZ (US)

(72) Inventors: Jerome LACOMBE, Phoenix, AZ (US); Matthew BARRETT, Phoenix, AZ (US); Frederic ZENHAUSERN, Phoenix, AZ (US)

(21) Appl. No.: 17/622,147

(22) PCT Filed: Jun. 26, 2020
(86) PCT No.: PCT/US2020/039944

§ 371 Date: Dec. 22, 2021

Related U.S. Application Data
(60) Provisional application No. 62/868,572, filed on Jun. 28, 2019.

(51) Int. Cl. C12M 1/12; C12N 5/071; C12N 5/077; C12M 3/00; C12M 1/32; C12M 3/06; C12M 1/00; G01N 21/64

(52) U.S. Cl. CPC C12M 25/04; C12N 5/069; C12N 5/0656; C12M 21/08; C12M 23/12; C12M 23/16; C12N 2513/00; C12M 23/34; C12M 23/38; C12M 29/10; C12M 29/04; G01N 21/6486; C12M 23/44

(57) ABSTRACT

A modular microfluidic device for simulating in vivo conditions of a biological system may include a first perfusion chamber having an inlet and an outlet, a second perfusion chamber having an inlet and an outlet, a well configured to hold one or more 3D structures of cultured biological cells. The well may be in fluid communication with the first and second perfusion chambers. A first porous membrane may be disposed between the first perfusion chamber and the well. A second porous membrane may be disposed between the second perfusion chamber and the well. The well may be configured to facilitate growth of cultured biological cells along all three dimensional axes, thereby providing or ensuring a more representative 3D structure of biological cells compared to conventional monolayer cultures.

### CROSS-REFERENCE TO RELATED APPLICATIONS

This application claims the benefit of priority to U.S. Provisional Patent Application No. 62/868,572 filed Jun. 28, 2019, which is hereby incorporated by reference in its entirety.

### BACKGROUND OF INVENTION

Many in vitro cellular studies are based on cell monolayer cultures and cannot satisfactorily mimic the complex three-dimensional environments and multi-component/constituent structure of biological tissue, including organs, tumors, and soft tissue. The status quo as it pertains to develop new anticancer drugs is to use monolayer (2D) cell culture or animal models in preclinical phase in order to assess efficacy, distribution and toxicity of the compounds. However, 2D cell culture does not recapitulate cell-cell interactions, cell-matrix interactions, nutrient and oxygen gradients, or cell polarity and, in addition to all ethical problems, animal models present important differences with human physiology. Hence, these models appear to be too limited and explain the important rate of failure of drug candidate in clinical phase while rising up the development cost.

Although some prior art multi-culture systems have been developed, they still suffer from the fundamental disadvantage of not reproducing the complete architecture of the modeled biological system. For example, the tumor microenvironment (TME) has a high impact on cancer cell radiation response and, ultimately, patient survival after radiation treatment. Accordingly, there is a need in the art for cell culture methods and systems that better reflect the actual 3-D properties, both physical and biological, of a biological tissue. In the context of cancer cell treatment, improved cancer cell culture systems that address these issues would be beneficial for a range of therapeutic and treatment applications.

### SUMMARY OF THE INVENTION

Provided herein are methods and systems that address the above problems by utilizing a modular microfluidic device for simulating in vivo conditions of a biological system. The modular microfluidic device may be configured to facilitate growth of the one or more 3D structures of cultured biological cells along all three dimensional axes. The methods and systems provided herein are compatible with a range of tissue cells, depending on the application of interest, such as cellular components of an organ, cancer cells of a tumor, cells of the vasculature, such as endothelial cells and smooth muscle cells, and combinations thereof. Any of the methods and systems provided herein may further comprise extracellular matrix, such as collagen, enzymes, and glycoproteins, that provide structural and biochemical support of surrounding cells.

In one embodiment, a modular microfluidic device for simulating in vivo dynamic conditions of a biological system comprises a first perfusion chamber having an inlet and an outlet, a second perfusion chamber having an inlet and an outlet, a well configured to hold one or more 3D structures of cultured biological cells and a lid. The well may be in fluid communication with the first and second perfusion chambers. The device may include a lid to seal the well while fluid is flowing through the first and/or second perfusion chambers. A first porous membrane may be disposed between the first perfusion chamber and the well. A second porous membrane may be disposed between the second perfusion chamber and the well. The well may be configured to facilitate growth of the one or more 3D structures of cultured biological cells along all three dimensional axes.

In one embodiment, a modular microfluidic device for simulating in vivo conditions of a biological system may include a first channel member having a first perfusion chamber formed therein, a second channel member having a second perfusion chamber formed therein, and a central member disposed between the first channel member and the second channel member. The first perfusion chamber may have an inlet and an outlet. The second perfusion chamber may have an inlet and an outlet. A first porous membrane may be disposed between the first perfusion chamber and the at least one well. A second porous membrane may be disposed between the second perfusion chamber and the at least one well. The central member may have a well formed therein, wherein the well is configured to facilitate growth of the one or more 3D structures of cultured biological cells along all three dimensional axes.

In one embodiment, the device may include a 3D structure of cultured biological cells disposed in the well of the central member. In one embodiment, the at least one well includes a first orifice in fluid communication with the first perfusion chamber via the first porous membrane, and a second orifice in fluid communication with the second perfusion chamber via the second porous membrane.

In one embodiment, an array of wells may be formed in the central member. In one embodiment, a first layer of living cells may be disposed on a surface of the first porous membrane. In one embodiment, the first layer of living cells comprises endothelial cells.

In one embodiment, each well of the array includes a first orifice in fluid communication with the first perfusion chamber via the first porous membrane, and a second orifice in fluid communication with the second perfusion chamber via the second porous membrane.

In one embodiment, a second layer of living cells may be disposed on a surface of the second porous membrane. In one embodiment, the second layer of living cells comprises cells selected from the group consisting of: fibroblast cells, mesenchymal cells and adipocyte cells.

In one embodiment, the central member comprises a translucent material. In one embodiment, the central member comprises an optically clear material.

In one embodiment, the first and second channel members comprise a white material. In some embodiments, the white material may allow luminescence reading of the device. For example, the top and bottom of the well containing the 3D cellular structures may be clear to allow the reading, but the chambers may be white to avoid luminescence diffusion.

In an alternative embodiment, the first and second channel members may comprise a black material. In some embodiments, the black material may allow luminescence reading of the device. For example, the top and bottom of the well containing the 3D cellular structures may be clear to allow the reading, but the chambers may be black to avoid luminescence diffusion.

Of course, the methods and systems provided herein are not constrained to any particular colors, but instead may be characterized as being reflective or absorbent with respect to wavelength ranges of the electromagnetic radiation, including in the visible range.

In one embodiment, the central member comprises polymers, preferably hard plastic materials such as polycarbonate. In one embodiment, the first and second porous membranes comprise track-etched polycarbonate. In other embodiments, the central member and channel members may comprise another injection-moldable biocompatible material. In one embodiment, the first channel member may comprise one or more alignment pins and the second channel member may comprise one or more alignment holes configured to receive the alignment pins.

In one embodiment, the first porous membrane is surrounded by a first membrane frame, and the second porous membrane is surrounded by a second membrane frame

In one embodiment a method of simulating in vivo conditions for a biological system may comprise: flowing a first fluid through a first perfusion chamber of a modular microfluidic device; passing a portion of the first fluid, via a first porous membrane, from the first perfusion chamber to a well containing one or more 3D structures of cultured biological cells; flowing a second fluid through a second perfusion chamber of the modular microfluidic device; passing a portion of the second fluid, via a second porous membrane, from the second perfusion chamber to the well; and three dimensionally growing the one or more 3D structures of cultured biological cells in the well.

In one embodiment, the method may include applying a first layer of living cells to a surface of the first porous membrane. In one embodiment, the method may include applying a second layer of living cells to a surface of the second porous membrane. In one embodiment, the first layer of living cells comprises endothelial cells. In one embodiment, the second layer of living cells comprises cells selected from the group consisting of: fibroblast cells, mesenchymal cells and adipocyte cells. The methods and systems provided herein are compatible with any number or types of components, such as cells, tissue and/or biofluids, including whole blood and constituents of whole blood, saliva, effusions, etc.

In one embodiment, the method may comprise the step of microscopically and/or spectroscopically imaging the one or more 3D structures of cultured biological cells. Microscopically imaging may include placing the device under an imaging microscope or spectroscope, and microscopically visualizing the one or more 3D structures of cultured biological cells through the device.

In one embodiment, the method may include the step of absorbance plate reading the one or more 3D structures of cultured biological cells. The absorbance plate reading may include placing the device into an absorbance plate reader, and measuring absorbance of the one or more 3D structures of cultured biological cells inside the device.

In one embodiment, the method may include the step of fluorescence plate reading the one or more 3D structures of cultured biological cells. The fluorescence plate reading may include placing the device into a fluorescence plate reader; and, measuring fluorescence of the one or more 3D structures of cultured biological cells through the device.

In one embodiment, the method may include the step of luminescence plate reading the one or more 3D structures of cultured biological cells. The luminescence plate reading may include placing the device into a luminescence plate reader, and measuring luminescence of the one or more 3D structures of cultured biological cells through the device.

### BRIEF DESCRIPTION OF THE DRAWINGS

FIG. 1A is a photograph of a first embodiment of an assembled modular microfluidic device.

FIG. 1B is a photograph of a holder apparatus for holding the microfluidic device of FIG. 1A.

FIG. 1C is a photograph showing the modular microfluidic device in a state of disassembly, including a view of the four round-bottom wells of the central member and the two chambers.

FIG. 2A is an inversed bright field microscope image of spheroids (SKOV3 cells) seeded in a well of a microfluidic device.

FIG. 2B is an upright fluorescence (DAPI stain) microscope image of the spheroids of FIG. 2A.

FIG. 3 is a graph showing the dose-dependent cytotoxic effect of select withanolides on SKOV3 ovarian cancer cells.

FIG. 4 is a graph showing the dose-dependent cytotoxic effect of WFA and WD on SKOV3 ovarian cancer cells cultivated in a 2D cell arrangement.

FIG. 5 shows: (below) a graph showing the radiosensitizing effect of WFA and WD on ovarian cancer cells cultivated in in a 2D cell arrangement; and (above) a schematic overview of the experiment.

FIG. 6A shows: (below) images of immunofluorescence elicited in SKOV3 cells 1 h, 6 h and 24 h after irradiation taken using an Epifluorescence microscope at a magnification of 63×; and (above) a schematic overview of the experiment.

FIG. 6B shows a quantification of γ-H2AX foci at 1 h, 6 h and 24 h after irradiation in cells previously exposed to DMSO or WD.

FIG. 6C shows a quantification of 53BP1 foci at 1 h, 6 h and 24 h after irradiation in cells previously exposed to DMSO or WD.

FIG. 7 is a Western blot showing expression level of ATM, S1981pATM, DNA-PKcs, S2056pDNA-PKcs, XRCC4, RPA70 and RAD51 in SKOV3 cells cultivated in 2D.

FIG. 8 shows: (below) normalized volume of spheroids of SKOV3 ovarian cancer cells as treated with DMSO, WFA or WD at a concentration of 0.7 μM 1 h before being irradiated at 0 Gy or 4 Gy; and (above) a schematic overview of the experiment.

FIG. 9 is a schematic cross section of microfluidic device including a well disposed between the first and second perfusion chambers, the well having optical plastic lining the underside thereof.

FIG. 10 is a schematic cross section of the microfluidic device of FIG. 9, showing an ovarian spheroid disposed in the well.

FIG. 11 is a schematic cross section of the microfluidic device of FIG. 10, showing endothelial cells disposed on the first porous membrane, the first porous membrane disposed between the first perfusion chamber and the well.

FIG. 12 is a schematic cross section of the microfluidic device of FIG. 11, showing fibroblasts or mesothelial cells disposed on the second porous membrane, the second porous membrane disposed between the first and second perfusion chamber and the well.

FIG. 13 is a schematic plan view of the microfluidic device of FIG. 12, showing blood in the first perfusion chamber flowing co-currently with the peritoneal fluid flowing in the second perfusion chamber.

FIG. 14 shows a second embodiment of an assembled microfluidic device.

FIG. 15 is a photo of the microfluidic device of FIG. 14, including a lid.

FIG. 16 is a schematic showing the microfluidic device of FIGS. 14-15 in use to simulate physiological conditions of a biological system while simultaneously facilitating three dimensional reproduction of the cells a three dimensional cellular structure.

FIG. 17A shows ovarian cancer spheroids visualized by bright field microscopy after 1 and 8 days of culture within the microfluidic device. Scale bar=200 μm.

FIG. 17B shows a live/dead assay on spheroids after 8 days of culture within the microfluidic device. Scale bar=500 μm.

FIG. 17C shows HUVECs cells cultured on the microfluidic device membrane and visualized by DAPI (blue) and F-actin staining (green) before and after flow induction. Scale bar=30 μm.

FIG. 18 shows an isometric view of a 3D model of a partially-assembled third embodiment of a microfluidic device, including a first channel member and a central member.

FIG. 19 shows: (above) a side elevation view of the microfluidic device of FIG. 18; and (below) a side elevation view of the porous membrane and membrane frame of the microfluidic device.

FIG. 20 shows an end elevation view of the microfluidic device of FIGS. 18-19

FIG. 21A shows a perspective view of the porous membrane and membrane frame.

FIG. 21B shows a perspective view of the central member.

FIG. 21C shows a perspective view of the porous membrane and frame of FIG. 21A assembled with the central member of FIG. 21B.

FIG. 22 shows a central member of FIGS. 21B-21C with a first channel member assembled thereto.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

As used herein, “biological system” is used broadly herein and refers to an in vitro biological unit of an organism that is useful in a variety of applications, including modelling and testing systems. A biological system may correspond to an organ, or a portion thereof, including cellular components thereof. For example, a biological system may be a lung, liver, intestine, heart, brain, etc., or cells thereof, including cells associated with blood vessels (e.g., endothelial cells, smooth muscle cells, and the like). A biological system may be a collection of one or more organs, tissue and/or biological fluid (e.g., blood, plasma, interstitial fluid, etc.) organized to perform one or more biological functions. For example, a biological system may be the respiratory system, digestive system, cardiovascular system, endocrine system, immune system, etc. A biological system may include a biological abnormality such as cancer. Accordingly, a “vascularized” biological system refers to a network of conduits within the biological system that is capable, at least in part, of transporting required oxygen and nutrients to the biological cells and removing waste product from the biological cell. The exchange may be similar to how exchange occurs in blood vessels, such as by diffusion. The systems are, of course, compatible with bulk media control and/or biomechanical conditions, where the cultured cells are immersed in a cell culture fluid that can be replaced or supplemented as desired.

“Three dimensional structures” or “3D structures” of cultured biological cells refers to a collection of cultured cells joined together via cell adhesion wherein the collection of cells is many cells across in all three spatial dimensions. A 3D structure of cultured biological cells may be at least 10 cells across in all three spatial dimensions. A 3D structure of cultured biological cells may be at least 100 cells across in all three spatial dimensions. Accordingly, as used herein a monolayer of cultured cells is not considered a 3D structure of cultured biological cells. In some embodiments, a 3D structure of cultured biological cells is a tissue, an organ, or a cancer mass. A 3D structure of cultured biological cells can include any eukaryotic cells that are able to self-organize in order to form a 3D structure that influences the spatial organization of the cell surface receptors engaged in interactions with surrounding cells, and may also induce physical constraints on the cells. These spatial and physical aspects in 3D cellular structures affect the signal transduction from the outside to the inside of cells, and ultimately influence gene expression and cellular behavior in a manner closer to that of the in vivo environment than a standard cellular monolayer culture. The systems and methods provided herein are compatible with a range of biological cell sources, including cells that have not been cultured, so long as the seeded cells are capable of subsequent cell culture after being introduced to the microfluidic device.

The devices disclosed herein provide a platform technology mimicking human ovarian TME, including culture of 3D cancer structures in close contact with several tissue compartments and extracellular matrix, in dynamic flow condition, while having high-throughput capacity and a ready-to-use configuration for subsequent biological analysis. Indeed, the device is highly customizable for any type of cells. For example, the different chambers may be controlled independently allowing cell culture in different medium, flow condition (shear stress, flow rate, etc.), gas environment, etc. Moreover, the device may be directly compatible with most of the common experimental methods and thus ready-to-use for microscopy imaging or plate reader analysis.

In one embodiment, the tumor-on-chip devices disclosed herein combine the advantages of standard cell culture (human cells, easy to handle, low cost) and animals models (complex spatial organization, interaction of cells) in order to mimic in vitro the human in vivo TME. This approach is especially beneficial for the study of cancer with a specific TME, such as ovarian cancer of which TME includes not only vascular and stroma system but also the unique presence of peritoneal cavity which drives many factors responsible of toxicity, metastasis dissemination, etc.

In one example, the different perfused chambers provide functionality to mimic and control independently both the flow condition of vascular system and peritoneal cavity. In some embodiments, the device allows for flexible design workflow (numbers of replicates, conditions, high-throughput, etc.) and analysis with diverse bottom/top optical readers.

In one embodiment, a tumor-on-chip may grow ovarian cancer cells spheroids, surrounded by functional monolayer of endothelial cells and fibroblasts cells seeded on membranes and cultured in representative dynamic condition. The device is user friendly by being directly compatible with microscopes and microplate readers and allowing investigation of several biological samples at the same time. If desired, fasteners such as clips may be used to ensure a tight fluid seal while facilitating convenience as to accessing the different cell compartments. The devices and methods are compatible with a range of membranes, including membranes with different pores, such as different pore density and/or sizes. Any type of cell can be used with the membranes, so long as the cell is capable of adhering to the membrane and is compatible with the instant cell culture techniques.

### Example 1: Development of Ovarian Tumor-On-Chip to Assess Cytotoxic Effect of New Natural Anticancer Compounds

The high number of anti-cancer drug candidates that fail to advance past the preclinical research stage is due, at least in part, to the lack of efficient in vitro models that can recapitulate in vivo human tumor microenvironment (TME) including 3D structure, multi-cellular components and dynamic flow environment. That is particularly true for some cancers, such as ovarian cancer, with a complex and specific molecular and cellular TME. The interaction of the TME with cancer cells plays a pivotal role in tumor development, progression and drug resistance (Worzfeld et al., 2017). Through the convergence of tissue engineering and microfluidics, small devices known as organs-on-a-chip have been engineered with the potential to improve the knowledge of such complex environments (Zhang et al., 2018).

Recently, cancer-on-a-chip models have emerged as a tool to study the TME. They contain small chambers for cell culture, enabling control over local gradients, fluid flow, tissue mechanics, and composition of the local environment (Tsai et al., 2017). Prior art cancer-on-a-chip systems still have several limitations. First, most of them implement co-culture using cancer cells and only one other cell type, thus neglecting other compartments such as vascular tissue, connective tissue or immune system. Second, the cancer-on-a-chip systems of the prior art don't provide for a three dimensional cell growth and can't culture spheroids/organoids in contact with other cell types. Third, these prior art cancer-on-a-chip systems are complex and arduous to set up and work with, and thus do not allow high-throughput analysis and/or are not directly compatible with common analytical methods. Hence, there is a need to develop a new platform enable simulation of the human TME, recapitulating the 3D structure of tumors and the main cellular compartments while allowing easy access for subsequent required biological analysis.

Provided herein are in vitro platforms that can mimic in vivo human organ microenvironment via modular microfluidic devices for simulating physiological conditions of biological systems, in order to provide new models for biomedical research. The microfluidic devices disclosed herein may be used to assess the efficacy of new natural compounds (e.g. withanolides) as cytotoxic agents for ovarian cancer. It is believed that 3D structure and multiple cellular components of TME widely influence ovarian cancer response to drug treatment. This is demonstrated herein via data regarding the difference of the efficacy of anti-proliferative drugs as applied to monolayer (2D) cellular structures as compared with spheroids/organoids or organ-on-chip cell culture. The tumor-on-chip microfluidic devices disclosed herein provide a new approach to assess cytotoxic activity of anticancer drugs on both cancer and normal cells and thus recapitulate the real in vivo effect of the drug in the ovarian TME.

In one embodiment of the present invention, the microfluidic device allows separation of interconnected chambers for culturing, in dynamic mode, three different tissue compartments of the TME: cancer spheroids (epithelial cancer cells), vascular tissue (endothelial cells) and connective tissue (fibroblasts).

Without wishing to be bound by theory, it is believed that the half-maximal inhibitory concentration (IC50) of withanolides is a function of the three dimensional nature of tumor structures and cell interactions therein. The tumor-on-chip microfluidic devices disclosed herein mimic the ovarian TME and are suitable for assessing cytotoxic effects of anti-cancer drugs in a simulated in vivo condition. Thus, the devices herein shed light on important questions in ovarian cancer such as immunotherapy resistance/toxicity or the role of peritoneal fluid in metastasis dissemination.

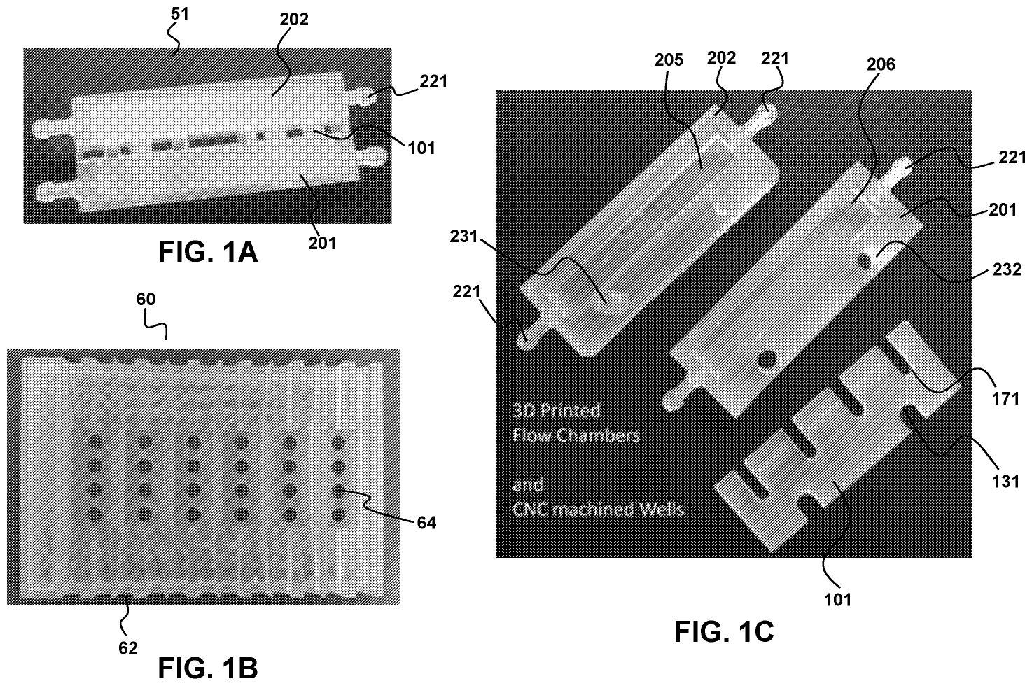

As shown in FIGS. 1A-1C, the tumor-on-chip microfluidic device 51 may comprise a central member 101 containing an array of four round-bottom wells 171 flanked by a first channel member 201 and a second channel member 202. The first channel member 201 includes a first perfusion chamber 205. The second channel member 202 includes a second perfusion chamber 206. The channel members have barbed connections 221 to serve as respective inlets/outlets for the perfusion chambers 205, 206, within the channel members. Each chamber 205, 205 is separated from the wells 171 of the central member 201 by a porous membrane (not shown) to allow exchanges between the chambers and wells 171. The central member 101 may comprise clear plastic to allow direct visualization of the contents of the wells 1771 under microscope. The device 51 may include alignment pins 231, disposed on the second channel member 202, and corresponding alignment holes 232, disposed on the first channel member 201, configured to securely receive the alignment pins. The central member 101 may include alignment grooves 131 configured to allow the alignment pins 231 to pass therethrough upon assembly of the device.

FIG. 1B shows a 96-well plate format holder apparatus 60. The holder apparatus functions to support and align six modular microfluidic devices, such as exemplary microfluidic device 51 shown in FIG. 1A. The holder apparatus 60 may include access holes 64 to allow access to the wells. The holder apparatus 60 may include barb collars 62 configured to receive the barbs of device 51 and thereby hold the device 51 in place in the holder apparatus.

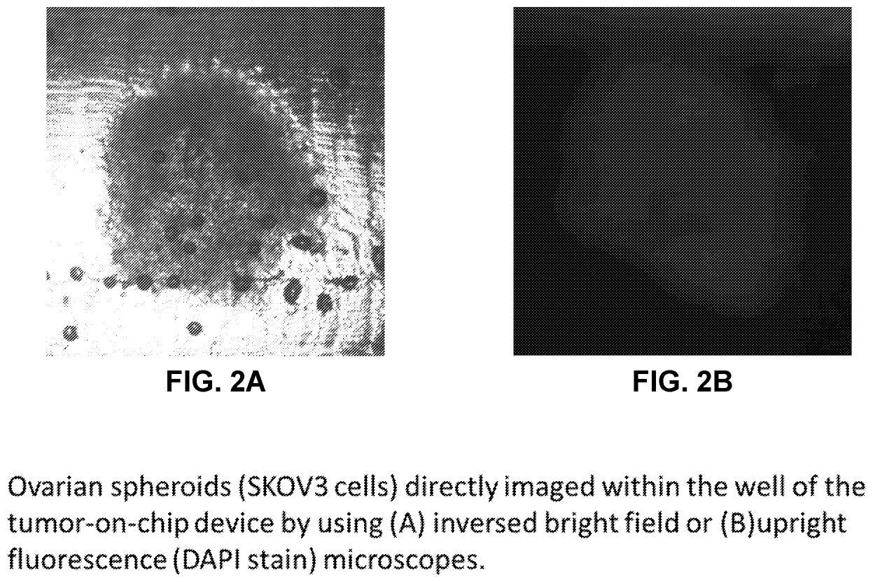

The modular microfluidic devices 51 may be configured to allow direct analysis in a plate reader. For example, the distance between each well in the strip may be configured to be compatible with the spacing of a 96-well plate. Furthermore, the microfluidic devices may be comprised of a clear or translucent material. Thus, as shown in FIG. 2A, spheroids seeded in the wells of the microfluidic are visible under bright field with inversed microscope. Furthermore, the spheroids are visible via upright fluorescence, as shown in FIG. 2B. Additionally, the spheroids are visible via MTT in some embodiments.

The device 51 may be configured for easy opening and to provide access to cells seeded on the membranes. Plastic material may be configured to have improved optical transparency to improve microscopic observation. The devices are useful for the simultaneous culture of multiple different cell types with different geometries, including 3-D dimensional configurations in the well (e.g., spheroids associated with certain tumors) and monolayers (e.g., endothelial cells associated with blood vessels and fibroblasts or mesothelial cells associated with the peritoneal fluid)

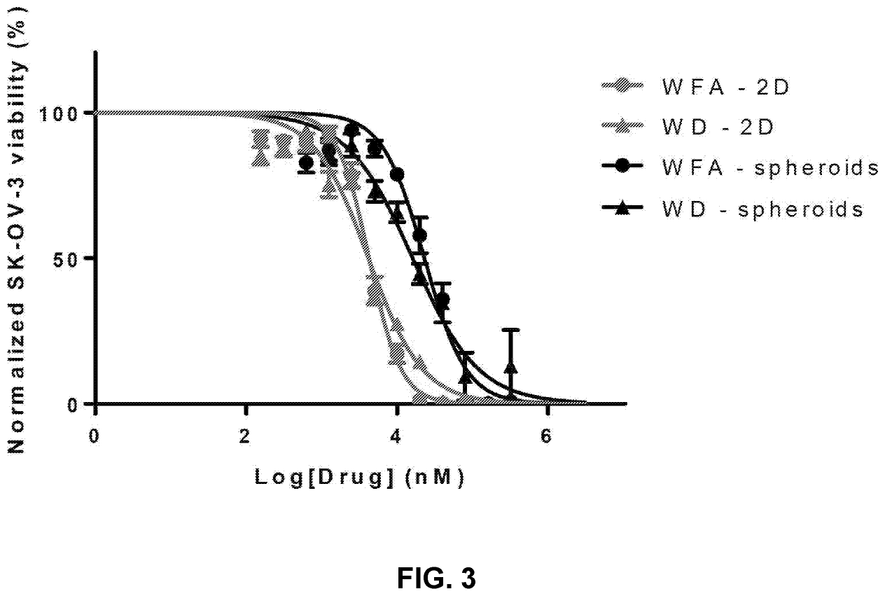

The withanolides withaferin A (WFA) and withanolide D (WD) were investigated for cytotoxicity when used to treat SKOV3 ovarian cancer cells. As shown in FIG. 3, WFA and WD have a cytotoxic effect on the SKOV3 ovarian cancer cell line. Unexpectedly, when assessed on SKOV3 spheroids, WFA and WD still have a significant antiproliferative effect but IC50 values increased from 4.4 μM and 4.2 μM to 23 μM and 16 μM, respectively. Thus, the results suggest that SKOV3 spheroids are more resistant to WFA and WD than SKOV3 cells cultured in monolayer and confirms that 3D structure plays an important role in cellular response to drug treatment.

As shown in FIG. 3, anti-cancer drugs such as Withanolides, including withaferin A (WFA) and withaferin D (WD), can have a dose-dependent cytotoxic effect on SKOV3 ovarian cancer cells cultured both as standard cell monolayer or as spheroids. Twenty thousand cells were seeded in an ultra-low affinity round bottom 96 well plates and 5 days were allowed for spheroids formation. Spheroids and cells in monolayer were then exposed to a large range of WFA or WD concentration (0.156 to 80 μM) for 48 h. Antiproliferative activity of WFA and WD have been assessed using MTT. Error bars represent mean±SEM from 3 independent experiments of at least 6 replicates each.

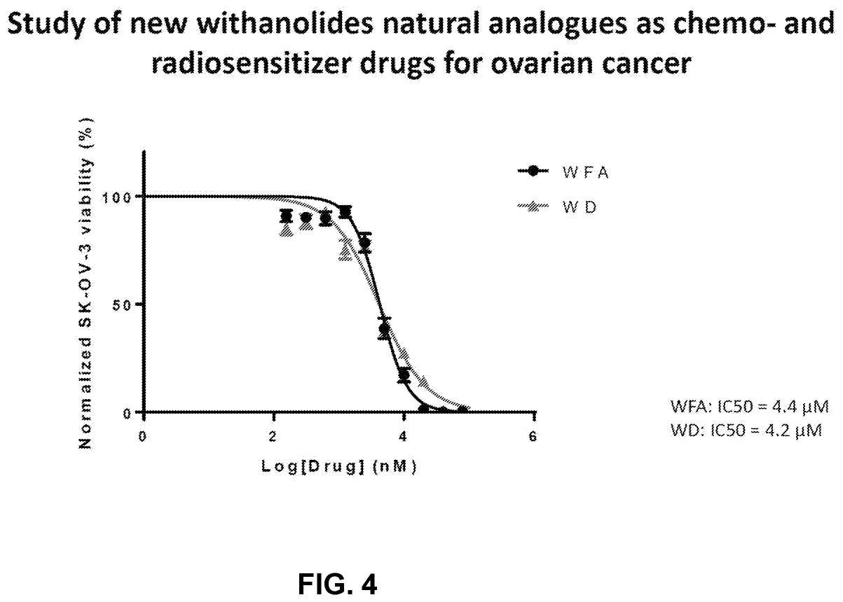

As shown in FIG. 4, WFA and WD have a dose-dependent cytotoxic effect on SKOV3 ovarian cancer cells cultivated in a 2D cell arrangement. Five thousand cells were seeded in a 96 well plate and exposed to a large range of WFA or WD concentration (0.156 to 80 μM) for 48 h. Antiproliferative activity of WFA and WD have been assessed using MTT. Error bar represent the mean±SEM from 3 independent experiments with at least 6 replicates each.

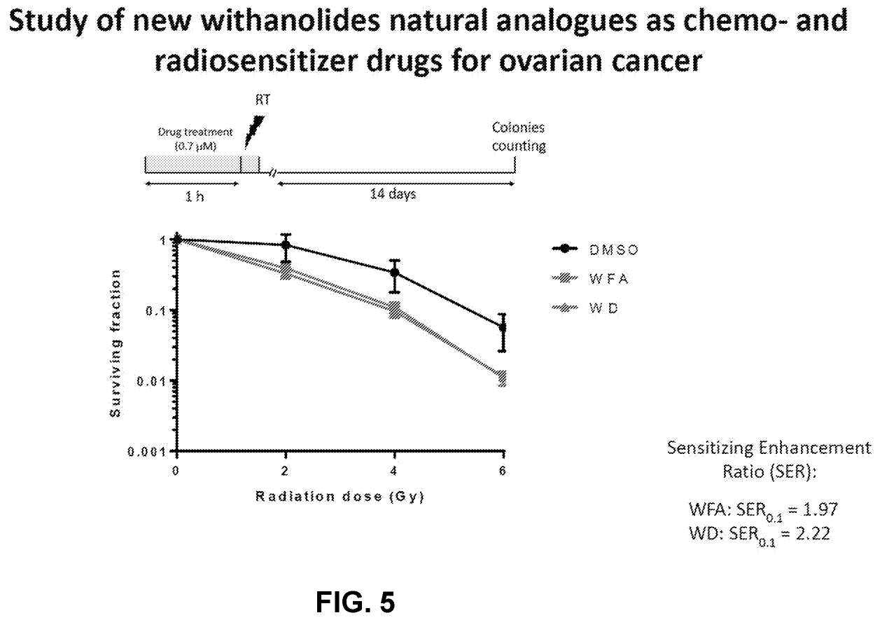

As shown in FIG. 5, WFA and WD have a radiosensitizing effect on ovarian cancer cells cultivated in a 2D cell arrangement. The radiosensitizing effect was assessed with a clonogenic assay. Cells were exposed to DMSO, WFA or WD at a concentration of 0.7 μM 1 h before irradiation and then were irradiated at 0, 2, 4 or 6 Gy. The medium was removed right after irradiation and replaced by a drug-free medium, changed twice a week for the rest of the experiment. Clonogenic assay was stopped when colonies containing at least 50 cells were observed under the microscope. Error bar represent mean±SEM from 3 independent experiments with at least 2 replicates each.

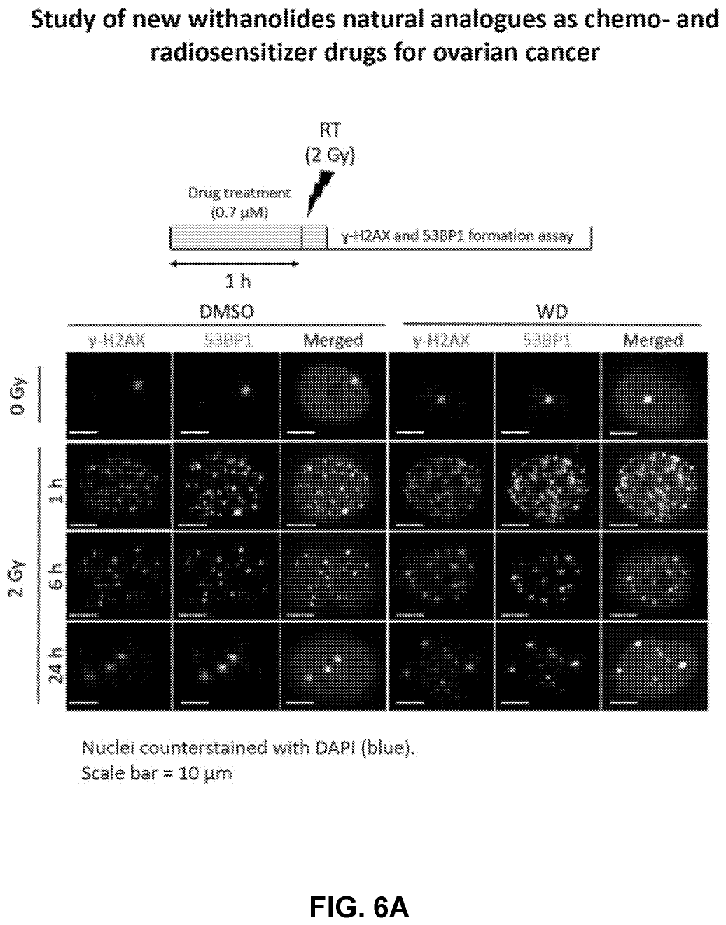

As shown in FIGS. 6A-6C, Monolayer SKOV3 cells treated with WD 1 h before irradiation have more DNA DSBs over 24 h than those exposed to DMSO compared to the non-irradiated cells. Cells were seeded on coverslip at a density of 5×104 per mL and exposed to DMSO or WD at a concentration of 0.7 μM 1 h before being irradiated at 0 or 2 Gy. The medium was removed and replaced by a drug-free medium immediately after irradiation. FIG. 6A. Images of immunofluorescence elicited in SKOV3 cells 1 h, 6 h and 24 h after irradiation taken using an Epifluorescence microscope at a magnification of 63×. Nuclei are counterstained with DAPI (blue), while γ-H2AX fluorescence is displayed in red and 53BP1 in green. Scale bar=10 μm.

FIG. 6B shows a quantification of γ-H2AX foci at 1 h, 6 h and 24 h after irradiation in cells previously exposed to DMSO or WD. Values are normalized with non-irradiated cells, exposed to DMSO or WD respectively. All the data are presented as the mean±SEM of at least 60 nuclei. *Significantly different values as determined by Student's t-test (p<0.05).

FIG. 6C shows a quantification of 53BP1 foci at 1 h, 6 h and 24 h after irradiation in cells previously exposed to DMSO or WD. Values are normalized with non-irradiated cells, exposed to DMSO or WD respectively. All the data are presented as the mean±SEM of at least 60 nuclei. *Significant differences as determined by Student's t-test (p<0.05).

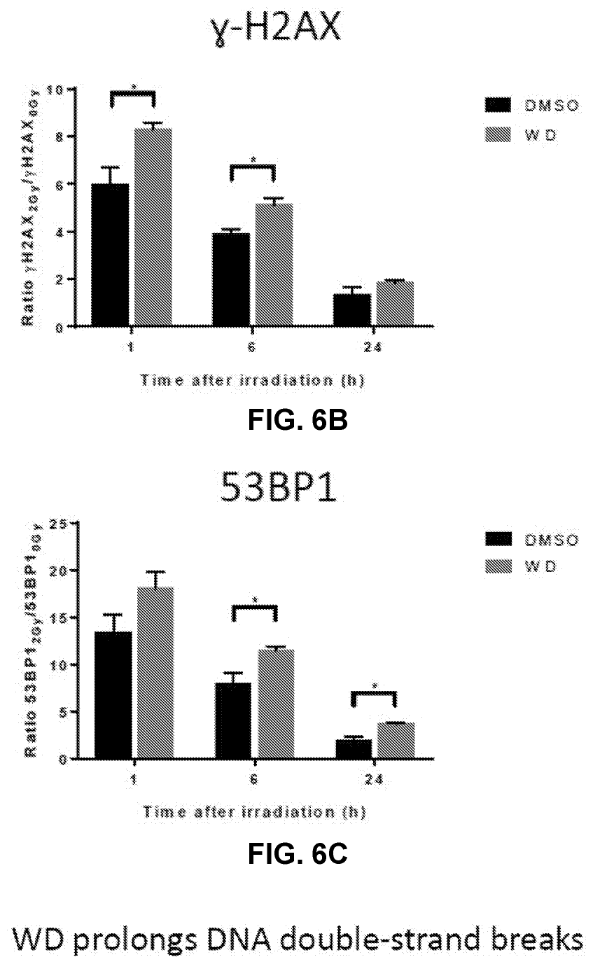

As shown in FIG. 7, WD radiosensitizes SKOV3 ovarian cancer cells by inhibiting the NHEJ DNA repair pathway. Western blot showing expression level of ATM, S1981pATM, DNA-PKcs, S2056pDNA-PKcs, XRCC4, RPA70 and RAD51 in SKOV3 cells cultivated in 2D. Cells were exposed to DMSO or WD at a concentration of 0.7 μM 1 h before undergoing an irradiation at 0 or 2 Gy. Proteins within cells were extracted 1 h, 6 h or 24 h after irradiation. A total of 5 μg of proteins was loaded. Bands intensities were quantified using ImageJ, normalized with GAPDH. Numbers represent expression level compared to non-irradiated cells. NI=Non-irradiated samples.

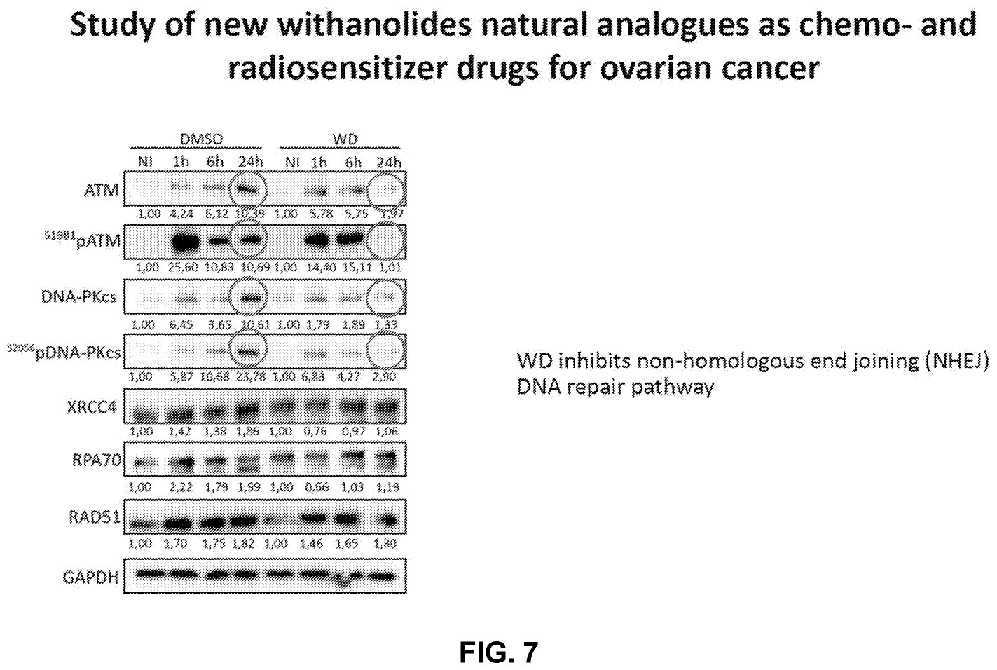

As shown in, FIG. 8, WFA and WD do not radiosensitize spheroids of SKOV3 ovarian cancer cells. FIG. 8 includes a schematic overview of experiment. Cells were plated in an ultra-low affinity 96 well plate with a round bottom and allowed to form spheroids for 5 days. At day 0, spheroids were treated with DMSO, WFA or WD at a concentration of 0.7 μM 1 h before being irradiated at 0 Gy or 4 Gy. The medium containing drug was replaced by a drug-free medium after 4 days, then freshly changed twice a week for the rest of the experiment. Diameter of each spheroid was measured two or three times a week for 19 days with Micrometrics SE Premium software. Spheroid volume growth over time. Volume were normalized to day 0. Error bars represent mean±SEM for at least 5 replicates.

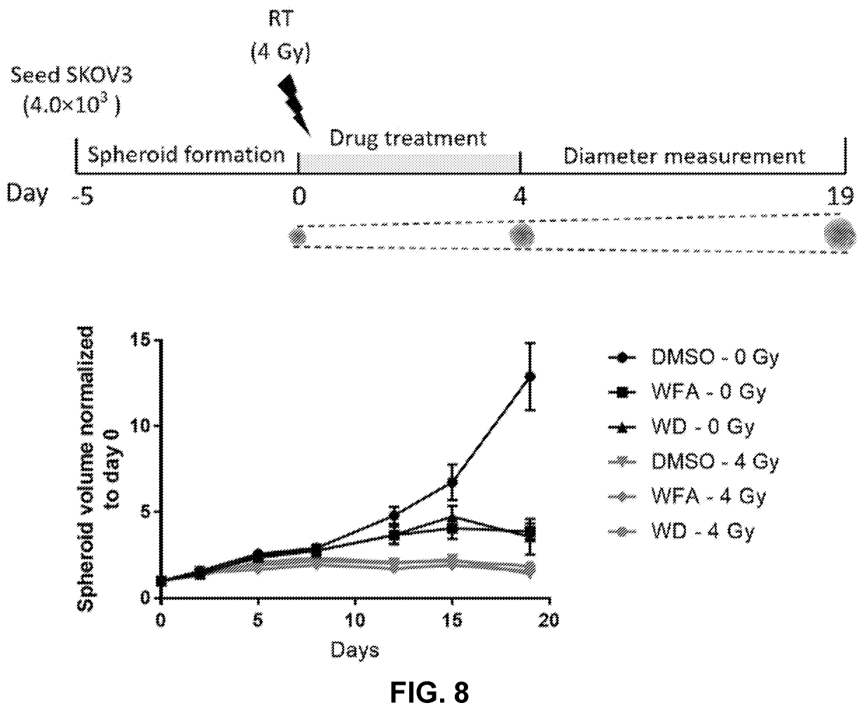

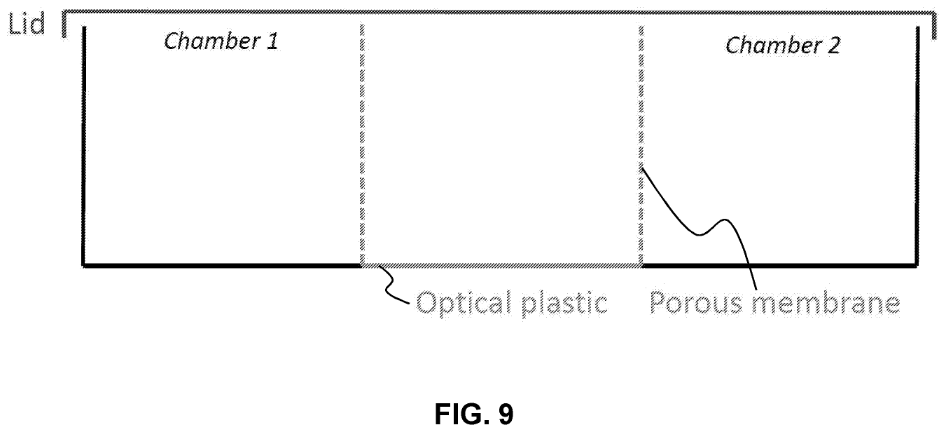

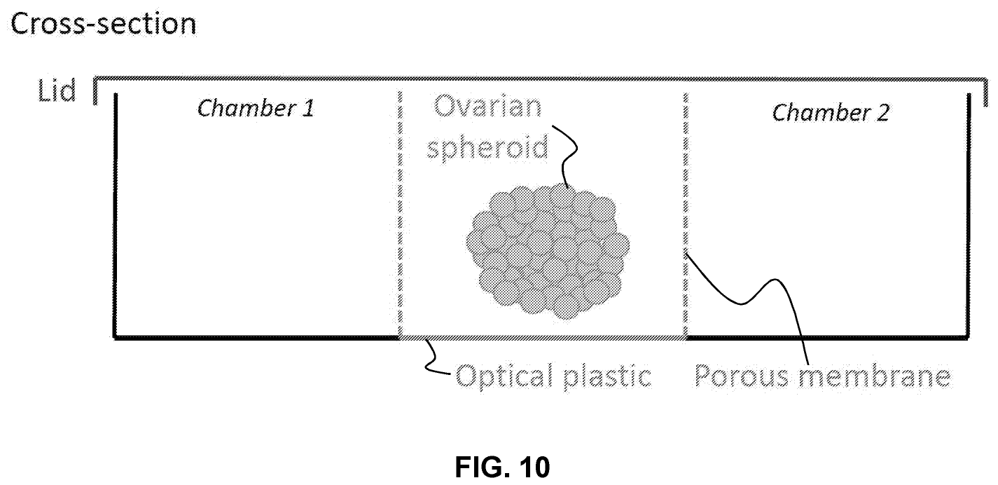

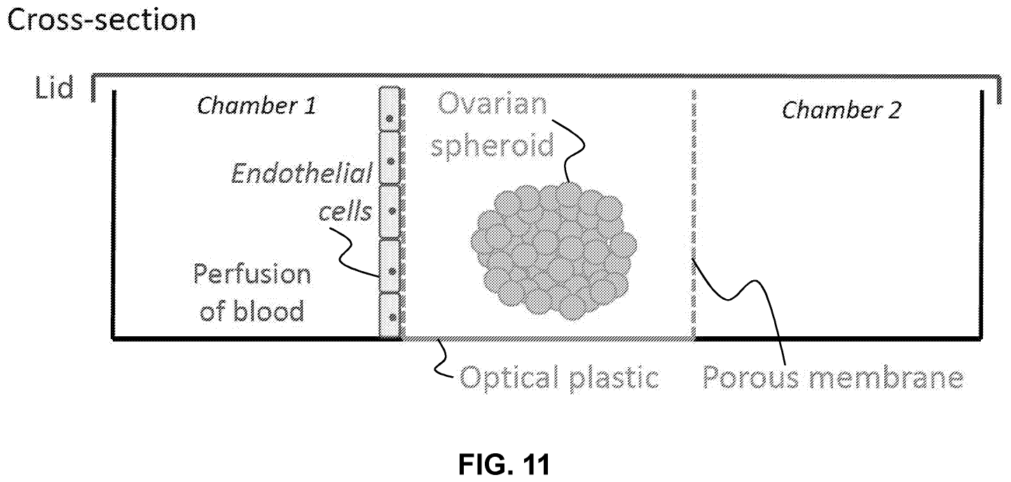

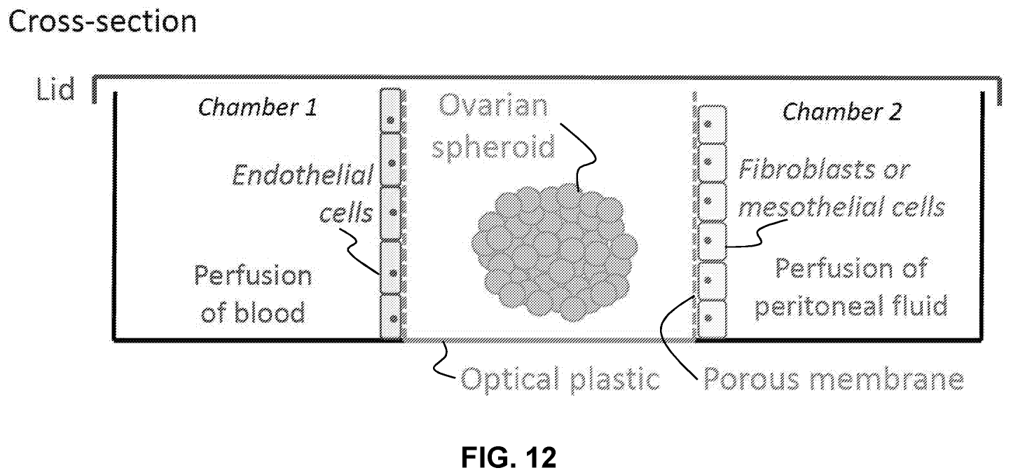

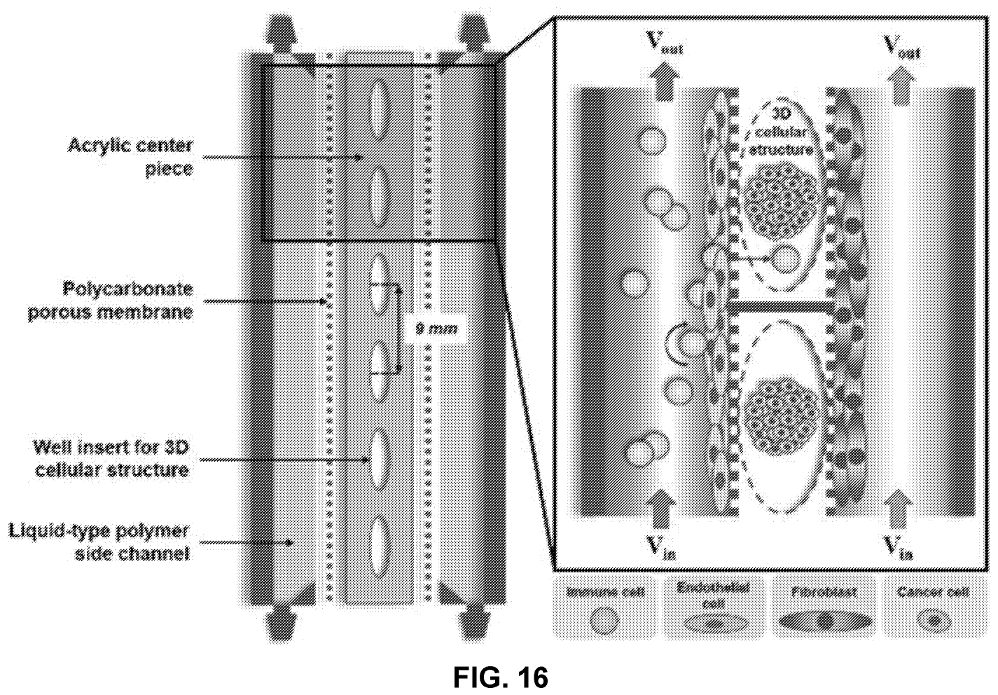

FIGS. 9-13, 16 schematically illustrate one embodiment of the process of configuring the device to simulate the TME. As shown in the schematic cross section of the microfluidic device in FIG. 9, the well (middle) is disposed between the first and second perfusion chambers, the well having optical plastic lining the underside thereof. As shown in FIG. 10, an ovarian spheroid may be disposed in the well. As shown in FIG. 11 endothelial cells may be disposed on the first porous membrane, between the perfusion of blood and the ovarian spheroid. As shown in FIG. 12, fibroblasts or mesothelial cells may be disposed on the second porous membrane, between the perfusion of peritoneal fluid and the ovarian spheroid. As shown in FIG. 13, blood in the first perfusion chamber may be configured to flow co-currently with the peritoneal fluid flowing in the second perfusion chamber. Of course, the devices and systems provided herein are compatible with a counter-current flow configuration, where flow directions are in a directionally opposed configuration. FIG. 16 shows a schematic plan view of an embodiment of the device having 6 wells, each of which contains a three-dimensional cellular structure.

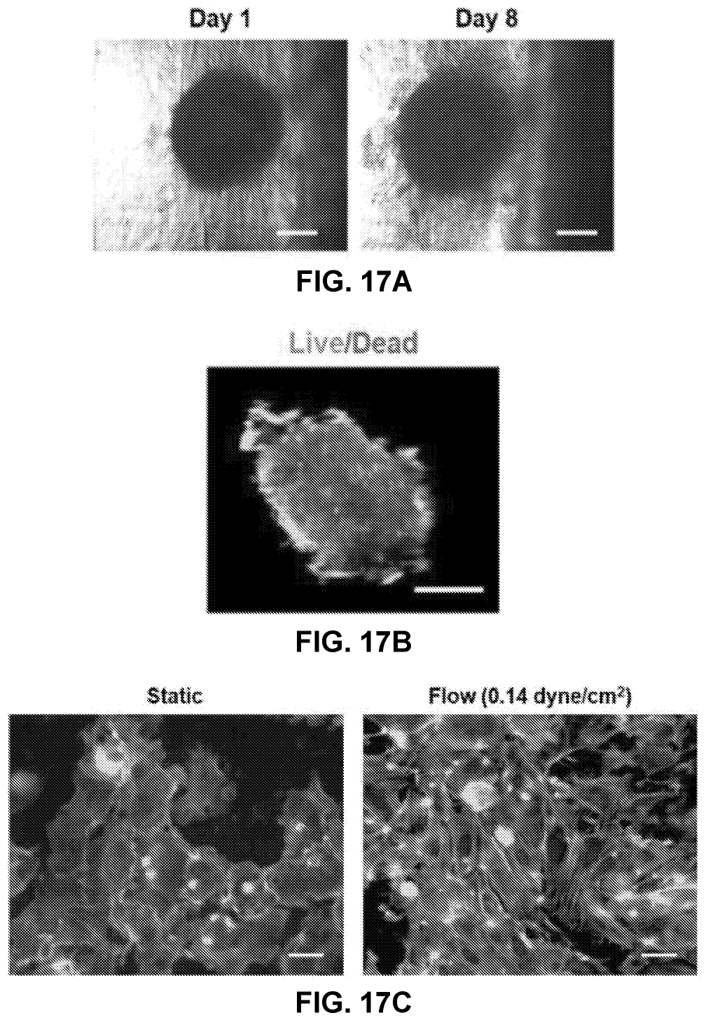

As shown in FIGS. 17A-C, in some embodiments, due at least in part to the device being comprised of transparent and or translucent materials, three dimensional cellular structures may be microscopically visualized within the device. FIG. 17A shows ovarian cancer spheroids visualized by bright field microscopy after 1 and 8 days of culture within the microfluidic device. Scale bar=200 μm. FIG. 17B shows a live/dead assay on spheroids after 8 days of culture within the microfluidic device. Scale bar=500 μm. FIG. 17C shows HUVECs cells cultured on the microfluidic device membrane and visualized by DAPI (blue) and F-actin staining (green) before and after flow induction. Scale bar=30 μm.

### Modular Microfluidic Tumor-On-Chip Design and Assembly

The device may be made with multiple materials, requiring several steps. The outer manifolds may be 3D printed using a Form2 SLA printer, creating the chambers 205, 206 and barbs 221 along with alignment pins 231 for assembly. The center member 101 may be machined Polycarbonate. The first and second channel members 201, 202 may be pressed together with double-sided medical PSA. Track-etched Polycarbonate porous membranes (8 μm pores) separate the chambers. Once the device 51 is assembled, it is cleaned with 70% ethanol and allowed to dry. Then, it is irradiated with X-Rays at 10 Gy (3 Gy/min) and kept in sterile condition until cell culture.

Cell Culture

Before proceeding to cell seeding, porous membranes were coated with collagen (50 μg/mL) for 4 hours. Endothelial HUVEC cells (ATCC® CRL-1730™) were first seeded on membrane into one of the chambers and allowed to attach for 2 hours. The device was then flipped 180° and Human Ovarian Fibroblast (HOF, Cat. #7330, ScienCell Research Laboratories) were seeded on the membrane of the other chamber and allowed to attach for 2 hours. After cell seeding, each chamber was perfused with respective medium (F-12K Medium (ATCC 30-2004) supplemented with 0.1 mg/mL heparin, 5 mL endothelial cell growth supplement (ECGS; BD Biosciences catalog #354006) and 10% of Fetal bovine serum (FBS) for HUVEC cells (ATCC® CRL-1730™) and Fibroblast Medium (FM, Cat. #2301, ScienCell Research Laboratories) for HOF cells) at a flow-rate of 10 μL/min for 7 days. In parallel SKOV3 cells (ATCC® HTB-77™) were maintained in McCoy's 5A medium supplemented with 10% of Fetal bovine serum (FBS) and 1% of antibiotics mixture streptomycin/penicillin at 37° C. in 5% CO2 atmosphere. The SKOV3 cells were seeded into a 96 round-bottom well ultra-low affinity plate at concentration of 10,000 cells/well and allowed to grow for 5 days. After 5 days, the resulting SKOV3 spheroids were transferred into the wells of the tumor-on-chip device. After the SKOV3 spheroids were loaded and HUVEC and HOF cells were seeded for a period of 7 days, the medium flowing through the HUVEC chamber was changed and replaced by medium-containing peripheral blood mononuclear cells (PBMCs). The PBMCs were previously isolated by using Ficoll gradient density medium from whole blood (healthy individual, BioChemed). PBMCs were perfused in the device for 1 day.

Cellular and Molecular Analysis

MTT assay was performed directly on spheroids, over a period of 5 days, to assess whether they were able to grow into the device. To assess cell communication and migration of PBMCs into the spheroids chambers, chemoattractant SDF-1a was injected into spheroids chambers and PBMCs were stained 24 hours later with CD2 antibody to detect, specifically, T-cells and NK cells, while the SKOV3 spheroids were stained with pan-cytokeratin (Abcam, ab7753). Cytokine profile was analyzed to investigate if secretion was modified by the presence of spheroids and/or fibroblasts. The device was disassembled at 2 and 7 days after initial HUVEC and HOF seeding. The porous membrane was removed to be analyzed using fluorescence analysis. Each porous membrane was cut into several pieces and was stained with α-tubulin (Santa Cruz, sc-5286), Pericentrin (abcam, ab4448), Connexin (Cell Signaling, P17302), Vimentin (abcam, ab92657), Ki67 (abcam, ab15580), β-catenin (Santa Cruz, sc-7963), α-catenin (abcam, ab51032), Ecadherin (ThermoFischer, 13-1700), VE-cadherin (Santa Cruz, sc-9989), Integrin β1 (Santa Cruz, sc-13590) and Phalloidins (Invitrogen, MAN0001777) antibodies to assess cell shape, cell junctions and cell proliferation. A full membrane was also stained with a live/dead cell viability assay to assess cell viability and distribution over the membrane.

Cytotoxicity Activity of Withanolides Compounds on Ovarian Tumor Microenvironment by Using Tumor-On-Chip.

The specifics of the TME may affect the cancer cells response to drug treatment. In this regard the effect of withanolides on SKOV3 speroids was investigated. It is believed that IC50 of withanolides is different when drugs are delivered to SKOV3 spheroids cultured in standard cell culture versus spheroids incubated in a tumor-on-chip device of the present disclosure. Thus, SKOV3 cells, cultured as: (1) a monolayer and (2) as spheroids in standard 96-well plates without contact with any other type of cells or on the tumor-on-chip, were exposed to different concentrations of WFA, WD and analogues as well as carboplatin/paclitaxel cocktail.

Experimental Protocol:

SKOV3 cells were maintained in the same condition as described above. Cells were seeded as: (1) a monolayer in 96-flat bottom well plates and as (2) spheroids in 96-round bottom well ultra-low affinity plates at a concentration of 5000 cells/well. The next day after cell seeding, the cells cultured as monolayer were exposed to a range of concentrations (0.1 to 80 μM) of WFA, WD, 15β-hydroxyWFA and 15β-hydroxyWD and a range of concentrations (20 to 320 μM and 3 to 50 μM) of carboplatin and paclitaxel, respectively, for 3 days. The cells cultured as spheroids were allowed to grow for 5 days before being exposed to the same drug concentrations for 3 days. Half of the spheroids were transferred into the tumor-on-chip before being exposed to the drugs following the same protocol previously described. The tumor-on-chip devices were previously seeded with HUVEC cells, HOF and PBMCs according to the same protocol described previously. After 3 days, the cells were exposed to MTT assay and absorbance was directly read from the plate and the tumor-on-chip on a BioTek® Epoch™ Microplate Spectrophotometer after overnight incubation at 37° C. For SKOV3 spheroids, another experiment was performed to measure growth rate over a long period of time. Spheroids were exposed to a unique dose of drug (to be determined according to IC50 curves) for 3 days and spheroids are then measured 2 or 3 times a week with a Nikon TS100-F microscope over a period of >20 days.

Statistical Analysis:

The estimation of IC50 values was performed in GraphPad Prism version 7.00 for Windows (GraphPad Software Inc., La Jolla, Calif.) using dose response curve fitting ((inhibition) vs normalized response (variable slope)) of SKOV3 cells treated with different doses of drugs. A Mann-Whitney test or Student's t-test was then used to compare IC50 of the different drugs according to the respective culture condition. p-value<0.05 was considered statistically significant. Each experiment was independently repeated 3 times with at least 4 internal replicates each time and expressed as a mean±standard error (SEM).

The 3D biological tumor cells on chip model was validated by showing the difference in cytotoxicity (as determined by IC50 values) of drugs exposed to SKOV3 cells cultured as (1) a monolayer and (2) unique spheroids or as spheroids into a co-culture device perfused with immune cells. This demonstrates that biological cells in the instant device are alive, responsive to internal and external stimuli. Furthermore, the devices and methods can facilitate testing of promising compounds, such as withanolides, for new anticancer drugs for ovarian cancer.

The devices and methods provided herein are compatible with a range of biological systems, thereby facilitating a range of modeling and testing applications, including non-cancer soft tissue test, such as drug efficacy, clearance and/or toxicity.

For example, mixed populations of mammalian and bacterial cells are compatible, with one compartment having mammalian cells and the other bacterial cells, thereby facilitating modeling of, and impact by, the microbiome.

### Example 2: Use of Modular Microfluidic Device to Study SABR Radiation Regimens and Damage to Normal Tissue

Despite the unheralded success of immune checkpoint blockade in delivering durable responses for some patients with non-small-cell lung cancer (NSCLC), the majority of patients do not respond to anti-programmed cell death protein 1 (PD-1) immune checkpoint inhibitors (ICIs) [1]. Yet, radiotherapy (RT) can increase the immunogenicity of cancer cells and can improve long-term antitumor responses when combined with ICIs [2]. However, there are still unknown practical considerations that may impact the efficacy of RT combined with immunotherapy, including the dose, fractionation and scheduling of RT with ICIs. In particular, stereotactic ablative RT (SABR) is a technological advancement that delivers higher doses of RT, using a hypofractionated schedule and with greater conformity. Thus, SABR reduces normal tissue toxicity and improves control of the primary tumor [3]. Nonetheless, evidence is required to elucidate whether SABR in combination with ICIs has superior immune-priming capability to conventional RT and study the optimal radiation regimen. The current lack of reliable data to investigate such mechanisms is mainly due to the inaccuracy of pre-clinical models that cannot reproduce the complexity of human tumor microenvironment (TME). Standard human in vitro models are not able to recapitulate the complete interaction of tumor-stroma-immune system and animal in vivo models suffer of important physiological differences. Hence, there is a critical need to exploit new bioengineered systems to accurately reproduce human TME and investigate its response to ionizing radiation in combination with immunotherapy.

The optimal SABR radiation regimen may be determined via the modular microfluidic devices disclosed herein. In one embodiment, the modular microfluidic devices maximize the lung tumor immune response while minimizing damages to normal tissue in combination with an anti-PD1 therapy by using a bioengineered platform recapitulating accurately human lung TME. The dynamic “tumor-on-chip” platform disclosed herein recapitulates the interaction of three-dimensional (3D) tumor structures with vascular compartment and the connective tissue. Published studies suggest that SABR may be more immunogenic than conventional RT [4,5].

In one embodiment, the modular microfluidic devices of the present disclosure allow determination of the optimal SABR radiation regimen to maximize NSCLC tumor response to anti-PD1 immunotherapy. Without wishing to be bound by theory, it is believed that a specific SABR regimen, to be determined as high single dose or hypofractionation, enhances tumor immunogenic cell death (ICD) and proimmunogenic inflammatory balance promoting anti-PD1 efficiency.

Furthermore, and it is believed that that the SABR regimen combined with anti-PD1 therapy may prevent damage to surrounding normal connective tissue compared to conventional RT.

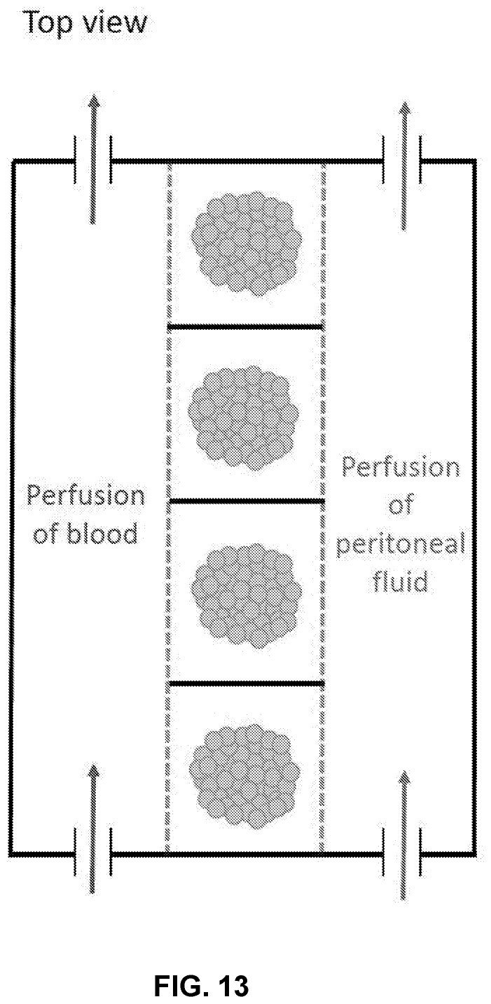

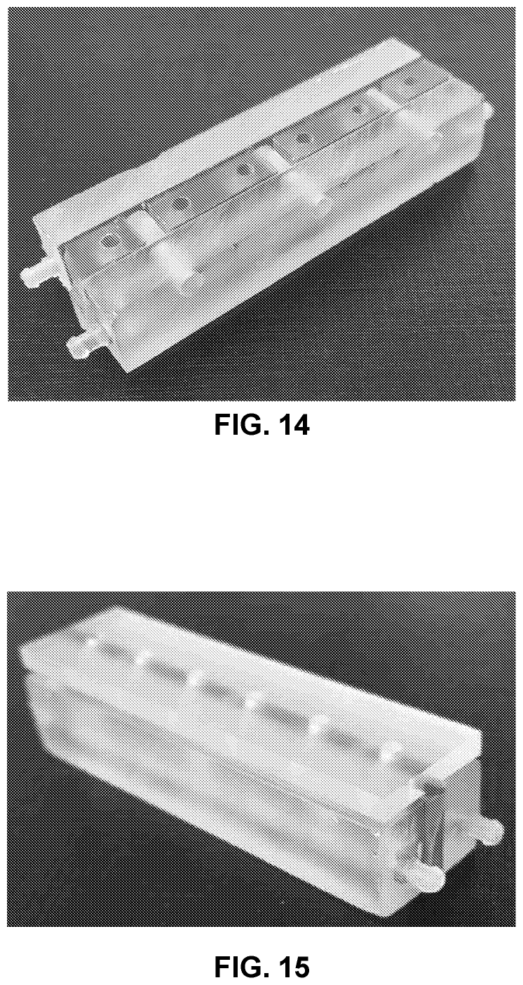

As shown in FIGS. 14-16, the modular microfluidic device includes a central member with six round-bottom well surrounded by two perfused chambers on each side. The modular microfluidic device may sometimes be referred to herein as an ASTEROIDS (Apparatus to Simulate Tumor Environment and Reproduce Organs by using an Interactive and Dynamic System). The central piece contains organoids/spheroids and is made with clear plastic to allow direct visualization under microscope. Each perfused chamber is separated from the central member by a porous membrane (8 μm pore size) to allow cellular and molecular exchanges between the chamber and well and on which cells can be cultured. The chambers being completely independent, different cell types with different growth condition can be cultured at the same time. Each chamber has a total volume of 66 mm3 and the membrane area is 165 mm2. A 96-well plate format holder (as in FIG. 1B) was also produced to lock up and align six strips simultaneously to be directly analyzed in a plate reader (distance (9 mm) between well in the central piece=the distance between well of a 96-well plate). The device can also be easily opened, thus offering access to chambers for subsequent analyses on cells seeded on membranes. Altogether, these features make the device a simple platform to study the human lung TME, offering a wide range of experimental setups to measure the desired biological outcomes.

Therefore, the device provides the appropriate platform to investigate the critical need of better understanding the effect of RT, and in particular SABR radiation regimen, on the lung TME immune response, when combined with immunotherapy such as anti-PD1 agent. The organ-on-chip technology may be combined with 3D cell culture to identification of the optimal SABR regimen to maximize the lung TME immune response to anti-PD-1 immunotherapy while minimizing damages to normal tissue

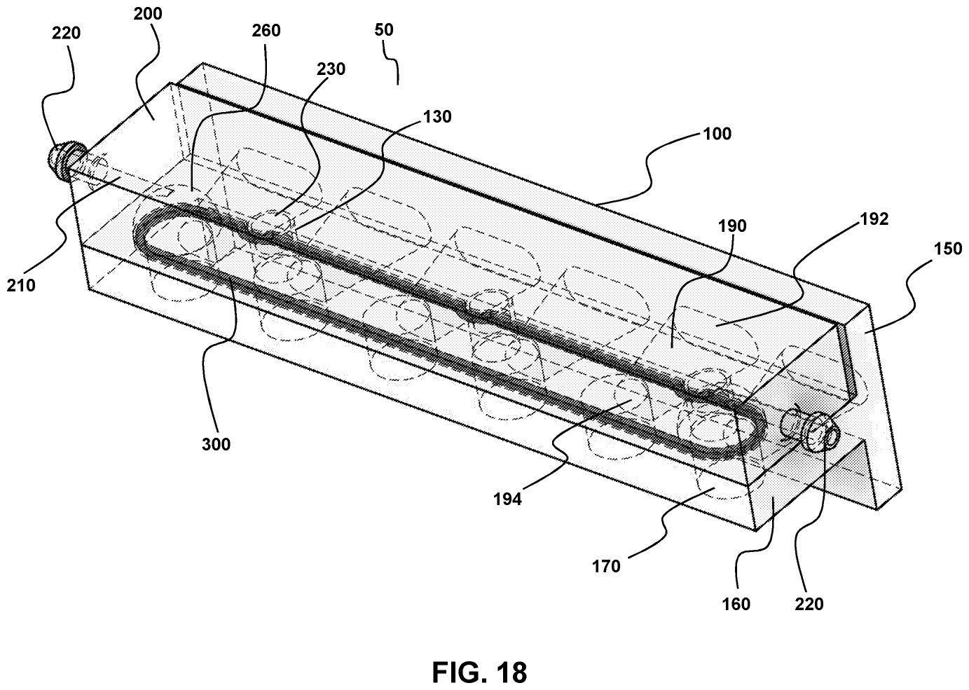

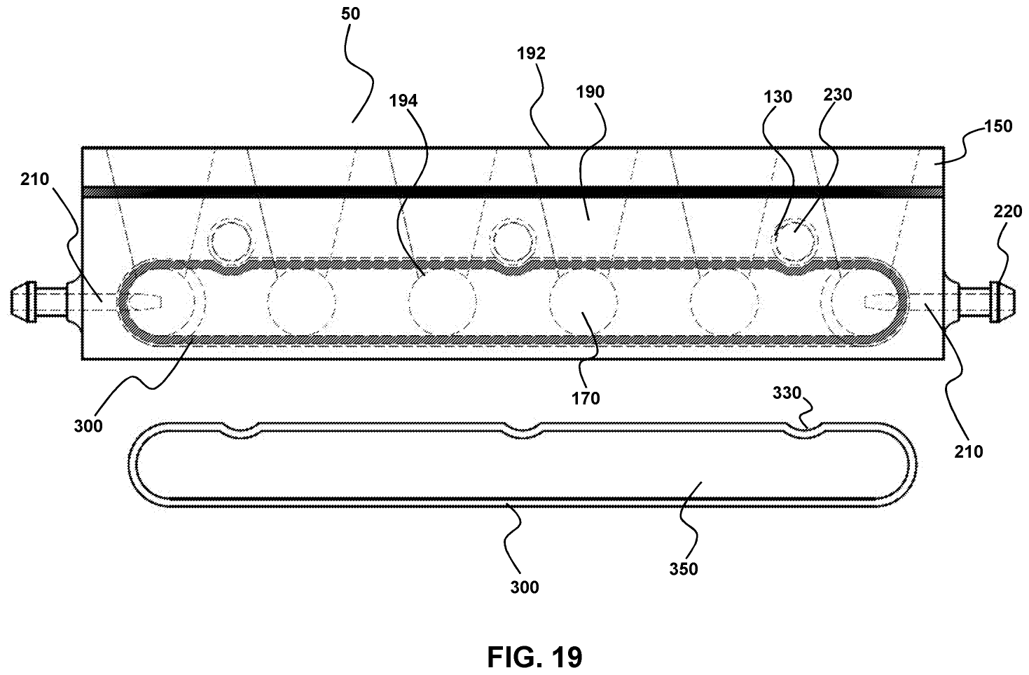

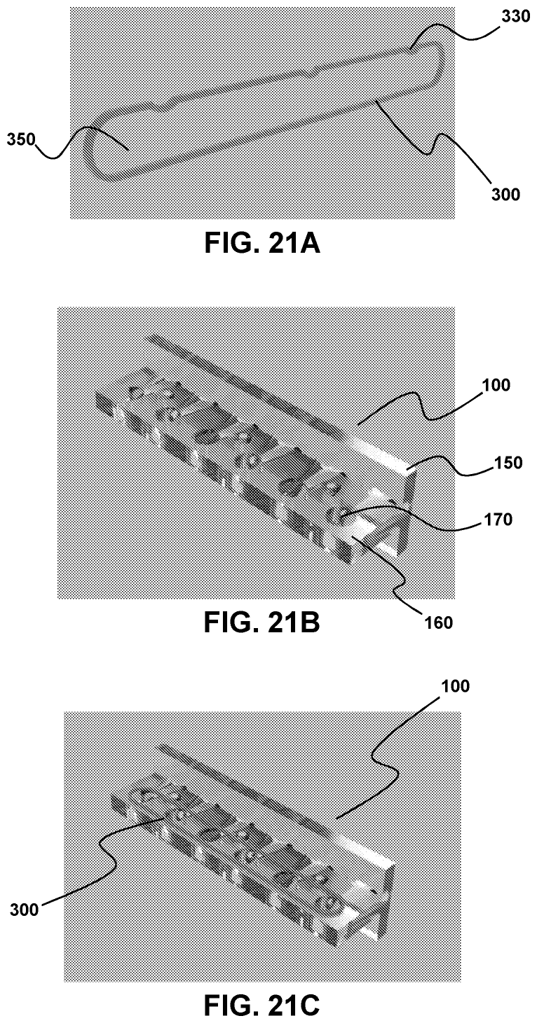

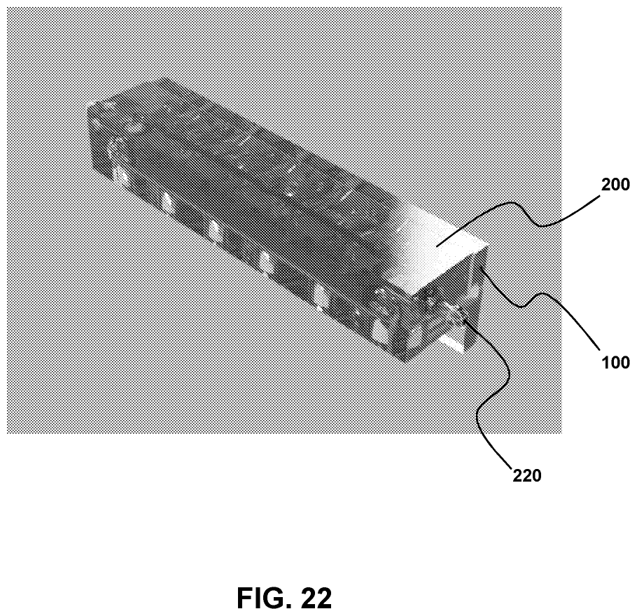

One embodiment of the modular microfluidic device is shown in FIGS. 18-22. For clarity, the device 50 of FIGS. 18-22 is shown with only the first channel member 200. It should be noted that in use, a second channel member (not shown) may be attached to the central member 100. Turning now to FIG. 18, the device is shown lying on its side. In the illustrated embodiment, the central member 100 includes a flange portion 150 and web portion 160. When the device is upright, the flange portion 150 runs along the top of the device, providing access to the wells 170 below. The web portion 160 includes an array of six wells 170 formed therein. The illustrated wells 170 comprise a round hole running all the way through the web portion 160, thus the wells may be said to have a first orifice facing the first channel member 200 and second orifice facing the other side of the device, where the second channel member (not pictured) attaches.

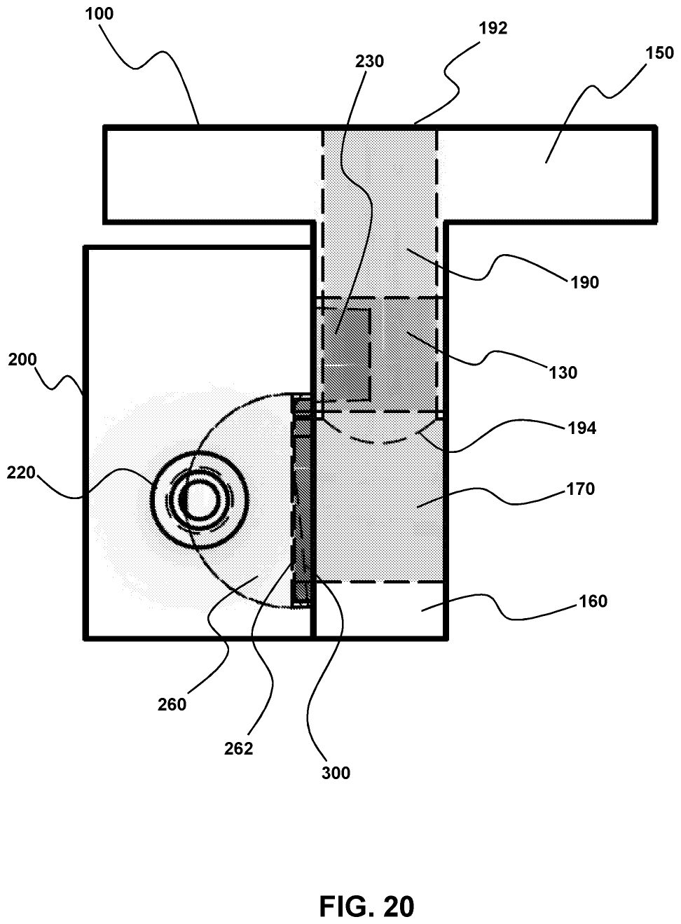

Device 50 may include a porous membrane 350 supported and surrounded by a membrane frame 300. The membrane frame 350 may be sandwiched securely in place between the channel member 200 and the web portion 160 of the central member 100. The channel member 200 may include a depression 262 (best shown in FIG. 20) formed therein to accept the membrane frame 300 and hold it in place. Thus, the porous membrane 300 cover the array of 6 wells 170 and is disposed between each of those wells and the perfusion chamber of the channel member 200. In use, the device may include a second porous membrane (not pictured) sandwiched between the web portion 160 of the central member 100 and the second channel member (not pictured), on the opposite side of the web portion from the first channel member 200.

Additionally, the web portion 160 may include well access ports 190 formed therein, providing access to the ports 170 from above. A bottom edge 194 of the access port 190 terminates in the well. A top edge 192 of the access port terminates on the upper surface of the flange portion 150.

The first channel member 200 includes barbed connections 220 at each end to facilitate the flow of perfusion fluid. The barbed connections may include flow distributors 210. The flow distributors 210 may terminate inside the perfusion chamber in a distributor throat 260. The illustrated distributor throat 260 is hemispherical. Of course, in other the embodiments the distributor throat may be another shape. The central member 100 may include alignment pins 130 on the web portion 160 in order to align and secure the first channel member 200 in place. In this regard, the first channel member 200 may include alignment holes 230 configured to snugly receive the alignment pins 130.

The modular microfluidic device 50 may be assembled and functionalized via a multi-step process. First, the channel member 200 may be 3D printed using a Form2 SLA printer, creating the large chamber and barbs 220 along with alignment pins 130 for assembly. In parallel, the central member 100 may be machined in acrylic material. Channel member 200 may be then sealed to central member 100 with double-sided medical PSA. Track-etched polycarbonate porous membranes 350 (8 μm pores) may separate the channels from the wells 170. Once the device is assembled, it may be cleaned with 70% ethanol and let dry. Then, it may be irradiated with X-Rays (>500 Gy) and kept in sterile condition until use.

Once assembled and before cell seeding, porous membranes may be functionalized with Collagen type I (50 μg/mL). After one-hour incubation, Human Lung Microvascular Endothelial Cells (HLMVECs) may be then seeded in one chamber. After cell attachment (˜1 h), the device may be flipped and normal lung fibroblasts (IMR-90) may be seeded in the other channel member. HLMVECs and IMR-90 may be cultured for 24 h in static condition until flow is initiated for 5 days. Flow rate in the vascular chamber may be 300 μL/min to generate a shear stress of 1.7 dyne/cm2, as usually encountered in tumor blood vessels [38]. Flow rate in the fibroblasts chamber may be 20 μL/min to reproduce cell surface shear stress (0.1 dyne/cm2) generated by interstitial flow within the TME [39].

In parallel, lung cancer cells (A549) may be grown in 96-well plates round bottom ultra-low attachment for 5 days to form spheroids. Initial numbers of cells may be adapted for each cell lines in order to obtain after 5 days spheroids with a size comprised between 0.5 and 1 mm. After 5 days, spheroids may be embedded in Matrigel (50 μL), loaded into the device and allowed to adapt for 24 hours. After spheroids loading, peripheral blood mononuclear cells (PBMCs) may be isolated by density gradient centrifugation from whole blood. PBMCs may be then resuspended in HLMVECs medium (2.106 cells/mL) and injected in the vascular compartment for 24 h before irradiation. After PBMCs injection, flow may be adjusted to 10 μL/min to match blood velocity in tumor blood vessels and favor extravasation and PBMCs infiltration.

Irradiation may be performed with a TrueBeam™ linear accelerator (Varian, Palo Alto, Calif.) utilizing a 6 MV energy, 600 MU/min dose rate, and the 120 Millennium multileaf collimator (MLC). An assembled ASTEROIDS unit, filled with a medium with similar density characteristics as the seeded cancer lines as described above, may be scanned using a Philips Brilliance CT Big Bore with a technique, scan length, and field of view appropriate to the device dimensions. A slice thickness of 1 mm may be utilized. The DICOM image dataset may be imported into the Varian Eclipse clinical treatment planning system. Treatment plans may be designed to deliver known amounts of radiation dose (see next following paragraph) to the target volumes using the AcurosXB 15.6.06 dose calculation algorithm and a 1 mm dose grid. Mobius3D software, which utilizes an independent beam model and dose calculation algorithm, may be used to perform an independent dose calculation to verify the accuracy of the treatment planning system dosimetry. The clinical daily quality assurance routine may be performed using a Sun Nuclear Daily QA™ 3 device (dose output verification) and Varian's integrated machine performance check (imaging, MLC, mechanical, and dose output verifications). The seeded device may be positioned on the TrueBeam linear accelerator's 6 degree of freedom treatment table. Onboard kV and MV imaging may allow for pre-treatment visualization. Small translational and rotational movements may be made by the table for accurate alignment of the device to within 1 mm. The designed radiation treatment plans may then be delivered.

The device may be irradiated at single dose 0, 8 and 24 Gy at a dose rate of 600 cGy per minute. To assess the effect of fractionation, 3×8 Gy (8 Gy/day for 3 continuous days, 24 Gy total dose) regimen may also be performed. For each data point, biological outcomes may also be measured in presence of anti-PD-1 antibody. To determine the most appropriate protocol for drug delivery, anti-PD-1 compound may be tested on A549 spheroids seeded in 96 well plates exposed to different anti-PD-1 concentration (0.001 to 10 μg/mL). The antibody may be delivered either 1 or 24 h before irradiation and injection may be pursued after irradiation at different intervals. Spheroids growth may be assessed by measuring spheroid size directly within the device every two days for 2 weeks under microscope. Volume of spheroids may be calculated using the formula 4/3π(d1+d2/4)³ where d1 is the length diameter and d2 the width diameter of the spheroid. The concentration and time of delivery may be chosen for the protocol that showed the minimal spheroids size after 2 weeks. Biological endpoints may be measured and analyzed at 2- and 7-days post-irradiation.

For each data points, three devices will be irradiated with 6 loaded spheroids in each of them. This sample size is determined for a two Independent sample study design with continuous endpoint by the equation n₁=(σ₁²+σ₂²/K)(z_{1-α/2}+z_{1-β})²/|μ₂−μ₁|² with an enrollment ratio K=n₁/n₂=1, assuming a probability of type-I error (a) of 0.05 and a risk of type-II error (β) of 0.2. σ₁ and σ₂ are the variance of anticipated mean μ₁ and μ₂ (extracted from published studies) and z is the critical Z value for a given α or β. A total of 48 devices may be irradiated for this aim (4 radiation regimen×2 drug concentration (with and without anti-PD-1)×2 timing points×3 replicates).

Analytic Methods

In order to estimate initiation of ICD, ATP and HMGB1 release and CRT membrane exposure may be assessed. ATP may be measured both in the supernatant and in the spheroids. Supernatant may be collected after irradiation from vascular compartment and connective tissue. Spheroids may be unloaded and analyzed in a 96 well plates. ATP assay may be performed using colorimetric ATP Detection Assay Kit (Abcam). Concentration of ATPₛₐₘₚₗₑₛ may be calculated from standard curve values and increase of ATP release may be assumed whether the ratio [ATP_{extracellular}]/[ATP_{intracellular}] of irradiated samples is higher than the ratio of sham-irradiated samples. In order to estimate the extracellular release of HMGB1, ELISA may be performed on supernatant by using the gold-standard HMGB1 ELISA Kit (Tecan). Release of HMGB1 may be assessed by comparing supernatant from irradiated samples to sham-irradiated sample in presence or not of anti-PD-1. Positive and negative controls available in the kit may be used to create standard curves and quantify released [HMGB1]. Finally, the presence of membrane CRT may be assessed by immunofluorescence microscopy (IF). Spheroids may be fixed with 0.25% paraformaldehyde but no permeabilized and incubated with primary anti-CRT antibody. After washing and incubation with fluorescence-labeled secondary antibody, fluorescence may be quantified by confocal microscopy. Exposure to secondary antibody alone may be used as a negative control.

To assess significance when comparing two groups, the Bartlett test may be first used to test if the tested biomarkers present significant homogeneous or heterogeneous variances between groups. Then, significance of two-group comparisons may be calculated using the Mann-Whitney nonparametric test (for heterogenous variances) or 2-tailed Student's t-test (homogeneous). P values of <0.05 may be considered significant.

In order to determine which radiation regimen may generate a proimmunogenic signature favorizing anti-PD-1 response, molecular signals on HLMVECs and A549 spheroids as well as secreted molecules I supernatant may be assessed. First, VCAM-1 may be detected by IF on HLMVECs attached on membrane. Second, NKG2DL and ICAM-1 expression may be assessed by IF on spheroids. Fluorescence signal intensity may be compared between membranes/spheroids exposed to different regimen by either nonparametric Mann-Whitney test or 2-tailed Student's t-test. Finally, TNF-α, IFN-α/β, IFN-γ, IL-1β, CXCL9, CXCL10 and CXCL16 may be simultaneously detected using Human Magnetic Luminex Assay (R & D systems) while TGF-β will be quantified by Human TGF-beta 1 Quantikine ELISA Kit (R & D systems) from collected supernatants. Cell culture media alone may be used as negative control. Cytokine values may be log-transformed and data may be first probed for normality using the Bartlett test.

Depending on result, either the nonparametric Mann-Whitney test or 2-tailed Student's t-test may be used to compare the individual cytokine level between two groups. A one-way ANOVA with Tukey's multiple comparison may be used to compare the effect between sham-, single and fractionated irradiated groups in presence or not of anti-PD-1 antibody. In order to assess which radiation regimen provides the best pro-immunogenic response, the combined cytokine performance may be calculated based on SVM analysis or receiver operating characteristic (ROC) curves. The generalized ROC criterion may find the best linear combination of cytokines such that the area under the ROC curve (AUC) is maximized. Viability of immune cells, fibroblasts, cancer cells and HLMVECs may be assessed by Live/Dead assays.

In addition to cytokine profile, PBMCs population may also be investigated. Indeed, TReg cells and tumor-associated macrophages have been associated with pro-tumor functions, whereas CD8+ T cells have been associated with anti-tumor functions[40-42]. Therefore, flow cytometry analysis may be performed on supernatant from spheroids chambers and connective tissue to assess immune cell infiltration depending on radiation regimen by using CD45+/CD25+/FOXP3+ staining (T_{Reg} cells), CD8+/CD4−/CD45+ staining (CD8+ T cells), CD45+/CD68+/CD80−/CD163+ staining (M2-macrophages). As negative control, samples incubated with secondary antibodies only may be used. Percentage of T_{Reg} cells, CD8+ t cells and M2 macrophages may be calculated according to the total number of infiltrating cells in a sample and compared between the different radiation regimen by either nonparametric Mann-Whitney test or 2-tailed Student's t-test.

Finally, spheroids growth will be monitored every day during the experiment duration by measuring spheroids size as described above in order to compare anti-PD-1 treatment efficiency in combination with the different radiation regimens.

To assess any toxicity from the different treatments, direct effect on normal fibroblasts may be assessed. First, radiation-induced DNA damages may be assessed by γH2AX/53BP1 foci assay. IMR90 may be collected at 2 and 7 days and stained for IF. Co-localized γH2AX/53BP1 foci from at least 100 cells from three independent experiments may be manually counted. Ratio of radiation-induced γH2AX/53BP1 foci (with the different radiation regimen) to sham-irradiated in presence or not of anti-PD-1 as well as absolute number of γH2AX/53BP1 foci for each condition may be calculated. Differences with control (sham-irradiated/sham-anti-PD-1 exposed) may be determined with Student t-test.

Second, IMR90 phenotype changes may be assessed to determine if the fibroblasts turn into a pro-inflammatory/pro-fibrotic phenotype [49] under certain radiation regimens. Thus, a series of experiments may be performed to monitor whether fibroblasts have been activated by any of the treatments. Gene expression assay of extracellular matrix & adhesion molecules (Qiagen, RT² Profiler™ PCR Array) may be performed to identify any potential changes in fibroblast secretory function, including overexpression of collagen and matrix metalloproteinase genes. Half of the membrane may be used to lyze directly the fibroblasts and mRNA will be extracted using RNA extraction kit (Qiagen) then qualitatively assessed with Bioanalyzer (Agilent). qPCR may be performed according to manufacturer protocol. Ct values may be normalized with five different housekeeping genes and analyzed according to delta Ct method [50] by comparing samples to sham-irradiated/sham-anti-PD-1 exposed sample. Gene expression may be significantly modified if the associated p-values are less than 0.05 and fold changes (FC) is greater than 1.5 or less than 0.66. In addition, collagen secretion may be assessed by total collagen assay (Abcam) in supernatant. Since activated fibroblasts also demonstrate cytoskeletal remodeling [51], vimentin and a-smooth muscle actin staining may be performed by IF as well as other activation biomarkers such as octamer-binding transcription factor-4 (Oct4), Nanog, collagen I, sex-determining region Y-box 2 (Sox2), chemokine receptor-4 (CXCR4), fibronectin. Proliferation biomarkers, including Ki67, may also be assessed. Finally, IMR90 senescence may be assessed by β-galactosidase assay (Cell Signaling). Membrane seeded IMR90 may be cut at the end of the experiment and put in IMR90 medium for 24 hours to perform in vitro assay. Senescence may be visualized by blue staining under bright field microscope. At least 100 cells (from 3 independent experiments) may be counted and t-test assay may be performed to determine if differences between samples and sham-irradiated/sham-anti-PD-1 exposed sample are significant. Negative control may be the sham-irradiated and sham-drug exposed device membrane. For positive control, IMR90 seeded on membrane may be exposed to etoposide (12.5 μM) for 24 hours and incubated 3 days prior imaging.

References Associated with Example 2
1. Kordbacheh T, Honeychurch J, Blackhall F, Faivre-Finn C, Illidge T. Radiotherapy and anti-PD-1/PD-L1 combinations in lung cancer: building better translational research platforms. Ann Oncol. 2018; 29: 301-310. doi:10.1093/annonc/mdx790
2. Frey B, Rückert M, Deloch L, RUhle P F, Derer A, Fietkau R, et al. Immunomodulation by ionizing radiation-impact for design of radio-immunotherapies and for treatment of inflammatory diseases. Immunol Rev. 2017; 280: 231-248. doi:10.1111/imr.12572
3. Baker S, Dahele M, Lagerwaard F J, Senan S. A critical review of recent developments in radiotherapy for non-small cell lung cancer. Radiat Oncol. 2016; 11: 115. doi:10.1186/s13014-016-0693-8
4. Lugade A A, Moran J P, Gerber S A, Rose R C, Frelinger J G, Lord E M. Local radiation therapy of B16 melanoma tumors increases the generation of tumor antigen-specific effector cells that traffic to the tumor. J Immunol Baltim Md 1950. 2005; 174: 7516-7523. doi:10.4049/jimmunol.174.12.7516
5. Marconi R, Strolin S, Bossi G, Strigari L. A meta-analysis of the abscopal effect in preclinical models: Is the biologically effective dose a relevant physical trigger? PLOS ONE. 2017; 12: e0171559. doi:10.1371/journal.pone.0171559
6. Golden E, Pellicciotta I, Demaria S, Barcellos-Hoff M H, Formenti S C. The convergence of radiation and immunogenic cell death signaling pathways. Front Oncol. 2012; 2. doi:10.3389/fonc.2012.00088
7. Demaria S, Ng B, Devitt M L, Babb J S, Kawashima N, Liebes L, et al. Ionizing radiation inhibition of distant untreated tumors (abscopal effect) is immune mediated. Int J Radiat Oncol Biol Phys. 2004; 58: 862-870. doi:10.1016/j.ijrobp.2003.09.012
8. Barker H E, Paget J T E, Khan A A, Harrington K J. The tumour microenvironment after radiotherapy: mechanisms of resistance and recurrence. Nat Rev Cancer. 2015; 15: 409-425. doi:10.1038/nrc3958
9. Wang Y, Deng W, Li N, Neri S, Sharma A, Jiang W, et al. Combining Immunotherapy and Radiotherapy for Cancer Treatment: Current Challenges and Future Directions. Front Pharmacol. 2018; 9. doi:10.3389/fphar.2018.00185
10. Zhang B, Korolj A, Lai B F L, Radisic M. Advances in organ-on-a-chip engineering. Nat Rev Mater. 2018; 3: 257-278. doi:10.1038/s41578-018-0034-7
11. Takebe T, Zhang B, Radisic M. Synergistic Engineering: Organoids Meet Organs-on-a-Chip. Cell Stem Cell. 2017; 21: 297-300. doi:10.1016/j.stem.2017.08.016
12. Lumniczky K, Candoias S M, Gaipl U S, Frey B. Editorial: Radiation and the Immune System: Current Knowledge and Future Perspectives. Front Immunol. 2017; 8: 1933. doi:10.3389/fimmu.2017.01933
13. Arnold K M, Flynn N J, Raben A, Romak L, Yu Y, Dicker A P, et al. The Impact of Radiation on the Tumor Microenvironment: Effect of Dose and Fractionation Schedules. Cancer Growth Metastasis. 2018; 11: 1179064418761639. doi:10.1177/1179064418761639
14. Segatto M, Tonini C, Pfrieger F W, Trezza V, Pallottini V. Loss of Mevalonate/Cholesterol Homeostasis in the Brain: A Focus on Autism Spectrum Disorder and Rett Syndrome. Int J Mol Sci. 2019; 20. doi:10.3390/ijms20133317
15. Portella L, Scala S. Ionizing radiation effects on the tumor microenvironment. Sem in Oncol. 2019; 46: 254-260. doi:10.1053/j.seminonco1.2019.07.003
16. Wersäll P J, Blomgren H, Pisa P, Lax I, Kälkner K-M, Svedman C. Regression of non-irradiated metastases after extracranial stereotactic radiotherapy in metastatic renal cell carcinoma. Acta Oncol Stockh Swed. 2006; 45: 493-497. doi:10.1080/02841860600604611
17. Siva S, MacManus M P, Martin R F, Martin O A. Abscopal effects of radiation therapy: a clinical review for the radiobiologist. Cancer Lett. 2015; 356: 82-90. doi:10.1016/j.canlet.2013.09.018
18. Lee Y, Auh S L, Wang Y, Burnette B, Wang Y, Meng Y, et al. Therapeutic effects of ablative radiation on local tumor require CD8+ T cells: changing strategies for cancer treatment. Blood. 2009; 114: 589-595. doi:10.1182/blood-2009-02-206870
19. Dewan M Z, Galloway A E, Kawashima N, Dewyngaert J K, Babb J S, Formenti S C, et al. Fractionated but not single-dose radiotherapy induces an immune-mediated abscopal effect when combined with anti-CTLA-4 antibody. Clin Cancer Res Off J Am Assoc Cancer Res. 2009; 15: 5379-5388. doi:10.1158/1078-0432.CCR-09-0265
20. Deng L, Liang H, Burnette B, Beckett M, Darga T, Weichselbaum R R, et al. Irradiation and anti-PD-L1 treatment synergistically promote antitumor immunity in mice. J Clin Invest. 2014; 124: 687-695. doi:10.1172/JCI67313
21. Dovedi S J, Adlard A L, Lipowska-Bhalla G, McKenna C, Jones S, Cheadle E J, et al. Acquired resistance to fractionated radiotherapy can be overcome by concurrent PD-L1 blockade. Cancer Res. 2014; 74: 5458-5468. doi:10.1158/0008-5472. CAN-14-1258
22. Sharabi A B, Nirschl C J, Kochel C M, Nirschl T R, Francica B J, Velarde E, et al. Stereotactic Radiation Therapy Augments Antigen-Specific PD-1-Mediated Antitumor Immune Responses via Cross-Presentation of Tumor Antigen. Cancer Immunol Res. 2015; 3: 345-355. doi:10.1158/2326-6066.CIR-14-0196
23. Liang H, Deng L, Chmura S, Burnette B, Liadis N, Darga T, et al. Radiation-induced equilibrium is a balance between tumor cell proliferation and T cell-mediated killing. J Immunol Baltim Md 1950. 2013; 190: 5874-5881. doi:10.4049/jimmuno1.1202612
24. Nichols D, Boyle T A, Noyes D, Latifi K, Feygelman V, Jackson I, et al. Evaluation of combined anti-PD-1 immunotherapy and radiation therapy in a preclinical mouse model of pneumonitis and fibrosis. J Thorac Dis. 2018; 10: 6254-6260. doi:10.21037/jtd.2018.10.01
25. Gong J, Le T Q, Massarelli E, Hendifar A E, Tuli R. Radiation therapy and PD-1/PD-L1 blockade: the clinical development of an evolving anticancer combination. J Immunother Cancer. 2018; 6: 46. doi:10.1186/s40425-018-0361-7
26. Xue J, Du S, Lu Y, Dicker A, Lu B. Abstract 3671: Anti-PD-1 treatment may potentiate the radiation-induced lung injury. Cancer Res. 2017; 77: 3671-3671. doi:10.1158/1538-7445.AM2017-3671
27. Myers C J, Lu B. Decreased Survival After Combining Thoracic Irradiation and an Anti-PD-1 Antibody Correlated With Increased T-cell Infiltration Into Cardiac and Lung Tissues. Int J Radiat Oncol Biol Phys. 2017; 99: 1129-1136. doi: 10.1016/j.ijrobp.2017.06.2452
28. Du S, Zhou L, Shukla G, Wang N, Yang L, Ma X, et al. Pharmacological Inhibition of PD-1 Exacerbates Radiation-Induced Cardiac Toxicity Through Cytotoxic T Cell-Mediated Myocarditis. Int J Radiat Oncol⋅Biol⋅Phys. 2016; 96: S88. doi: 10.1016/j.ijrobp.2016.06.221
29. Gubin M M, Zhang X, Schuster H, Caron E, Ward J P, Noguchi T, et al. Checkpoint blockade cancer immunotherapy targets tumour-specific mutant antigens. Nature. 2014; 515: 577-581. doi:10.1038/nature13988
30. Seidel J A, Otsuka A, Kabashima K. Anti-PD-1 and Anti-CTLA-4 Therapies in Cancer: Mechanisms of Action, Efficacy, and Limitations. Front Oncol. 2018; 8: 86. doi:10.3389/fonc.2018.00086
31. Jenkins R W, Barbie D A, Flaherty K T. Mechanisms of resistance to immune checkpoint inhibitors. Br J Cancer. 2018; 118: 9-16. doi:10.1038/bjc.2017.434
32. Wirsdörfer F, de Leve S, Jendrossek V. Combining Radiotherapy and Immunotherapy in Lung Cancer: Can We Expect Limitations Due to Altered Normal Tissue Toxicity? Int J Mol Sci. 2018; 20. doi:10.3390/ijms20010024
33. Asna N, Livoff A, Batash R, Debbi R, Schaffer P, Rivkind T, et al. Radiation therapy and immunotherapy—a potential combination in cancer treatment. Curr Oncol. 2018; 25: e454-e460. doi:10.3747/co.25.4002
34. Deloch L, Derer A, Hartmann J, Frey B, Fietkau R, Gaipl U S. Modern Radiotherapy Concepts and the Impact of Radiation on Immune Activation. Front Oncol. 2016; 6: 141. doi:10.3389/fonc.2016.00141
35. Formenti S C, Demaria S. Combining Radiotherapy and Cancer Immunotherapy: A Paradigm Shift. JNCI J Natl Cancer Inst. 2013; 105: 256-265. doi:10.1093/jnci/djs629
36. Golden E B, Apetoh L. Radiotherapy and Immunogenic Cell Death. Semin Radiat Oncol. 2015; 25: 11-17. doi:10.1016/j.semradonc.2014.07.005
37. Serrano-del Valle A, Anel A, Naval J, Marzo I. Immunogenic Cell Death and Immunotherapy of Multiple Myeloma. Front Cell Dev Biol. 2019; 7. doi:10.3389/fce11.2019.00050
38. Fisher D T, Muhitch J B, Kim M, Doyen K C, Bogner P N, Evans S S, et al. Intraoperative intravital microscopy permits the study of human tumour vessels. Nat Commun. 2016; 7: 10684. doi:10.1038/ncomms10684
39. Mitchell M J, King M R. Computational and Experimental Models of Cancer Cell Response to Fluid Shear Stress. Front Oncol. 2013; 3. doi:10.3389/fonc.2013.00044
40. Gajewski T F, Schreiber H, Fu Y-X. Innate and adaptive immune cells in the tumor microenvironment. Nat Immunol. 2013; 14: 1014-1022. doi:10.1038/ni.2703
41. Nishikawa H, Sakaguchi S. Regulatory T cells in cancer immunotherapy.

Curr Opin Immunol. 2014; 27: 1-7. doi:10.1016/j.coi.2013.12.005
42. Noy R, Pollard J W. Tumor-associated macrophages: from mechanisms to therapy. Immunity. 2014; 41: 49-61. doi:10.1016/j.immuni.2014.06.010
43. Shen R N, Hornback N B, Shidnia H, Lu L, Montebello J F, Brahmi Z. A comparison of lung metastases and natural killer cell activity in daily fractions and weekly fractions of radiation therapy on murine B16a melanoma. Radiat Res. 1988; 114: 354-360.
44. Schaue D, Ratikan J A, Iwamoto K S, McBride W H. Maximizing tumor immunity with fractionated radiation. Int J Radiat Oncol Biol Phys. 2012; 83: 1306-1310. doi:10.1016/j.ijrobp.2011.09.049
45. Matsuzawa-Ishimoto Y, Hwang S, Cadwell K. Autophagy and Inflammation. Annu Rev Immunol. 2018; 36: 73-101. doi:10.1146/annurev-immunol-042617-053253
46. Sgambato A, Casaluce F, Sacco P C, Palazzolo G, Maione P, Rossi A, et al. Anti PD-1 and PDL-1 Immunotherapy in the Treatment of Advanced Non-Small Cell Lung Cancer (NSCLC): A Review on Toxicity Profile and its Management. Curr Drug Saf. 2016; 11: 62-68. doi:10.2174/1574886311207040289
47. Graves P R, Siddiqui F, Anscher M S, Movsas B. Radiation pulmonary toxicity: from mechanisms to management. Semin Radiat Oncol. 2010; 20: 201-207. doi:10.1016/j.semradonc.2010.01.010
48. Tsoutsou P G, Koukourakis M I. Radiation pneumonitis and fibrosis: mechanisms underlying its pathogenesis and implications for future research. Int J Radiat Oncol Biol Phys. 2006; 66: 1281-1293. doi:10.1016/j.ijrobp.2006.08.058
49. Citrin D E, Prasanna P G S, Walker A J, Freeman M L, Eke I, Barcellos-Hoff M H, et al. Radiation-Induced Fibrosis: Mechanisms and Opportunities to Mitigate. Report of an NCI Workshop, Sep. 19, 2016. Radiat Res. 2017; 188: 1-20. doi:10.1667/RR14784.1
50. Livak K J, Schmittgen T D. Analysis of relative gene expression data using real-time quantitative PCR and the 2(−Delta C(T)) Method. Methods San Diego Calif. 2001; 25: 402-408. doi:10.1006/meth.2001.1262
51. Nomura S. Identification, Friend or Foe: Vimentin and a-Smooth Muscle Actin in Cancer-Associated Fibroblasts. Ann Surg Oncol. 2019; 26: 4191-4192. doi:10.1245/s10434-019-07894-8
52. Nickoloff J A, Boss M-K, Allen C P, LaRue S M. Translational research in radiation-induced DNA damage signaling and repair. Transl Cancer Res. 2017; 6: S875-S891. doi:10.21037/tcr.2017.06.02
53. Mariotti L G, Pirovano G, Savage K I, Ghita M, Ottolenghi A, Prise K M, et al. Use of the γ-H2A X Assay to Investigate DNA Repair Dynamics Following Multiple Radiation Exposures. PLOS ONE. 2013; 8: e79541. doi:10.1371/journal.pone.0079541

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A modular microfluidic device for simulating physiological conditions of a biological system, the device comprising:
a first channel member having a first perfusion chamber formed therein, the first perfusion chamber having an inlet and an outlet;
a second channel member having a second perfusion chamber formed therein, the second perfusion chamber having an inlet and an outlet;
a central member disposed between the first and second channels members; the central member comprising:
at least one well configured to facilitate growth of biological cells along all three dimensional axes into one or more 3D structures of cultured biological cells and/or maintain one or more 3D structures of biological cells in all three-dimensional axes; a first porous membrane disposed between the first perfusion chamber and the at least one well; and
a second porous membrane disposed between the second perfusion chamber and the at least one well.

2. The device of claim 1 comprising a 3D structure of cultured biological cells disposed in the well of the central member.

3. The device of claim 1, wherein the at least one well includes a first orifice in fluid communication with the first perfusion chamber via the first porous membrane, and a second orifice in fluid communication with the second perfusion chamber via the second porous membrane.

4. The device of claim 1 comprising an array of wells formed in the central member.

5. The device of claim 4, wherein each well of the array includes a first orifice in fluid communication with the first perfusion chamber via the first porous membrane, and a second orifice in fluid communication with the second perfusion chamber via the second porous membrane.

6. The device of claim 4, wherein the central member includes a well access passage for each well.

7. The device of claim 1 comprising:
a first layer of living cells disposed on a surface of the first porous membrane.

8. The device of claim 7, wherein the first layer of living cells comprises endothelial cells.

9. The device of claim 8 comprising:
a second layer of living cells disposed on a surface of the second porous membrane.

10. The device of claim 9, wherein the second layer of living cells comprises one or more cell types selected from the group consisting of: fibroblast cells, mesenchymal cells and adipocyte cells.

11-18. (canceled)

19. A modular microfluidic device for simulating physiological conditions of a biological system, the device comprising:
a first perfusion chamber having an inlet and an outlet;
a second perfusion chamber having an inlet and an outlet;
a well configured to hold one or more 3D structures of cultured biological cells, wherein the well is in fluid communication with the first and second perfusion chambers;
a lid to seal the well;
a first porous membrane disposed between the first perfusion chamber and the well;
a second porous membrane disposed between the second perfusion chamber and the well; and
wherein the well is configured to facilitate cell culture growth of biological cells along all three dimensional axes into the one or more 3D structures of cultured biological cells and/or maintain the one or more 3D structures in all three-dimensional axes.

20. A method of simulating physiological conditions for a biological system, the method comprising:
flowing a first fluid through a first perfusion chamber of a modular microfluidic device;
passing a portion of the first fluid, via a first porous membrane, from the first perfusion chamber to a well containing one or more 3D structures of cultured biological cells;
flowing a second fluid through a second perfusion chamber of the modular microfluidic device;
passing a portion of the second fluid, via a second porous membrane, from the second perfusion chamber to the well; and
three dimensionally growing the one or more 3D structures of cultured biological cells in the well.

21. The method of claim 20, further comprising:
applying a first layer of living cells to a surface of the first porous membrane, wherein the first layer of living cells comprises endothelial cells.

22. (canceled)

23. The method of claim 21, further comprising:
applying a second layer of living cells to a surface of the second porous membrane.

24. The method of claim 23, wherein the second layer of living cells comprises cells selected from the group consisting of: fibroblast cells, mesenchymal cells and adipocyte cells.

25. The method of claim 20, wherein the first fluid comprises blood or a constituent thereof.

26. The method of claim 20, wherein the second fluid comprises peritoneal fluid or a constituent thereof.

27. The method of claim 20, wherein the flow of the first fluid is co-current to the flow of the second fluid.

28-31. (canceled)

32. The method of claim 28, further comprising the step of luminescence plate reading the one or more 3D structures of cultured biological cells by:
placing the device into a luminescence plate reader; and,
measuring luminescence of the one or more 3D structures of cultured biological cells through the device.

33. The method of claim 20, further comprising the step of culturing a microbiome in the first or second perfusion chamber.

34-35. (canceled)
